SEQUENCE LISTING (58 sequences)

SEQ ID NO: 1 - DNA, 51 bp, Homo sapiens
agtggacccagaaactgcattggcaygaggtttgctctcatgaacatgaaa

SEQ ID NO: 2 - DNA, 50 bp, Homo sapiens
agttcagtgtctccatcacacccagygtagggccagctgcatcactggca

SEQ ID NO: 3 - DNA, 51 bp, Homo sapiens
ctaagaaaccaaattttaggaacttyttagtgctctccacaaaggggtctt

SEQ ID NO: 4 - DNA, 20 bp, artificial
  primer
aattctgctgtcagggcaac

SEQ ID NO: 5 - DNA, 25 bp, artificial
  primer
ttgagagaaagaatggatccaaaaa

SEQ ID NO: 6 - DNA, 21 bp, artificial
  primer
ggcatagagtctgcagtcagg

SEQ ID NO: 7 - DNA, 23 bp, artificial
  primer
tcaccttctatcacactccatca

SEQ ID NO: 8 - DNA, 20 bp, artificial
  primer
tcagtgtctccatcacaccc

SEQ ID NO: 9 - DNA, 23 bp, artificial
  primer
ggattgttgagagagtcgatgtt

SEQ ID NO: 10 - DNA, 22 bp, artificial
  primer
ttccatcagtcagtcccatcac

SEQ ID NO: 11 - DNA, 21 bp, artificial
  primer
caagttctggttgggaagagc

SEQ ID NO: 12 - DNA, 21 bp, artificial
  primer
gcaggatttcaatgaccagcc

SEQ ID NO: 13 - DNA, 22 bp, artificial
  primer
tgggcctaattgattctttggc

SEQ ID NO: 14 - DNA, 20 bp, artificial
  primer
ctgtcagaggggctagaggt

SEQ ID NO: 15 - DNA, 20 bp, artificial
  primer
cctcccaggttcaagcgatt

SEQ ID NO: 16 - DNA, 24 bp, artificial
  primer
gcatttagtccttgtgagcacttg

SEQ ID NO: 17 - DNA, 23 bp, artificial
  primer
catacgttctgtgtggggacaac

SEQ ID NO: 18 - DNA, 22 bp, artificial
  primer
acctgccttcaatttttcactg

SEQ ID NO: 19 - DNA, 20 bp, artificial
  primer
cttcactagcccgattctgc

SEQ ID NO: 20 - DNA, 22 bp, artificial
  primer
gtcacaatccctgtgacctgat

SEQ ID NO: 21 - DNA, 20 bp, artificial
  primer
ttggagacagcaatgaccgt

SEQ ID NO: 22 - DNA, 25 bp, artificial
  primer
ctgtttcactttgtagatatgggac

SEQ ID NO: 23 - DNA, 21 bp, artificial
  primer
aatcccactgggcctaaagac

SEQ ID NO: 24 - DNA, 20 bp, artificial
  primer
caactgcccttgcagcattt

SEQ ID NO: 25 - DNA, 20 bp, artificial
  primer
acccaggaagccagactttg

SEQ ID NO: 26 - DNA, 20 bp, artificial
  primer
tctgcccatctgtcaccaat

SEQ ID NO: 27 - DNA, 20 bp, artificial
  primer
ttggccacatgtccagtact

SEQ ID NO: 28 - DNA, 20 bp, artificial
  primer
ggcaccagataaccaccttc

SEQ ID NO: 29 - DNA, 22 bp, artificial
  primer
gggctctagattgacaaaaaca

SEQ ID NO: 30 - DNA, 22 bp, artificial
  primer
tcctccacacatctcagtaggt

SEQ ID NO: 31 - DNA, 20 bp, artificial
  primer
taaggcctgaccttgtccct

SEQ ID NO: 32 - DNA, 27 bp, artificial
  primer
acttcacgaatactatgatcatttacc

SEQ ID NO: 33 - DNA, 20 bp, artificial
  primer
cattgaccctttgggaatga

SEQ ID NO: 34 - DNA, 119 bp, Homo sapiens
ccacgtatgtaccacccagcttaacgaatgctctactgtcatttctaaccataatctctttaaagagctcttttgtctttcagtatctcttccctgtttggaccacattacccttcatc

SEQ ID NO: 35 - DNA, 16 bp, artificial
  primer
gcatcctgcaccacca

SEQ ID NO: 36 - DNA, 17 bp, artificial
  primer
ggatgaccttgcccaca

SEQ ID NO: 37 - DNA, 171 bp, Homo sapiens
gaaaaatgtggggtatctcttccctgtttggaccacattacccttcatcatatgaagccttgggtggctcctgtgtgagactcttgctgtgtgtcacaccctaatgaactagaacctaaggttgctgtgtgtcgtacaactagggaacgtatgaaggtcaactccctgtgc

SEQ ID NO: 38 - DNA, 39 bp, Homo sapiens
gaaaaatgtggggaacgtatgaaggtcaactccctgtgc

SEQ ID NO: 39 - DNA, 39 bp, Homo sapiens; identical to SEQ ID NO: 38

SEQ ID NO: 40 - DNA, 39 bp, Homo sapiens; identical to SEQ ID NO: 38

SEQ ID NO: 41 - DNA, 455 bp, artificial
  Misc_RNA
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtagagcattcgttaagcgttttagagctagaaatagcaagttaaaataaggctagtccgttatcaacttgaaaaagtggcaccgagtcggtgctttttttctagacccagctttcttgtacaaagttggcatta

SEQ ID NO: 42 - DNA, 455 bp, artificial
  Misc_RNA
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagggtaatgtggtccaaacgttttagagctagaaatagcaagttaaaataaggctagtccgttatcaacttgaaaaagtggcaccgagtcggtgctttttttctagacccagctttcttgtacaaagttggcatta

SEQ ID NO: 43 - DNA, 99 bp, artificial
  Misc_RNA
ccagcttaacgaatgctctactgtcatttctaaccataatctctttaaagagctcttttgtctttcagtatctcttccctgtttggaccacattaccct

SEQ ID NO: 44 - DNA, 2220 bp, Homo sapiens
gggcagggaagctccaggcaaacagcccagcaaacagcagcactcagctaaaaggaagactcacagaacacagttgaagaaggaaagtggcgatggacctcatcccaaatttggcggtggaaacctggcttctcctggctgtcagcctggtgctcctctatctgatgagagtccccaaatcagcgaagagtccacttactaaacagacataaggaaatgaagtgtcctggaagaattcctgcctgaacctctcaggagcatttgaggacatttatcaagtattcactccaggattgggactatgaagacttcagctgctttcagctaatcattgagacttttcaggggtctcagaatagtcaggaaaggacctgatgagtgaatgcaattactgatgttggagttgctgttattatttatcgtgtacatattacctccctctcttgaccattccagttcctgagtaactcaccagccctctgatctataaagtcacaatccctgtgacctgatttctgtttcactttgtagatatgggacccgtacacatggactttttaagagactgggaattccagggcccacacctctgcctttgttgggaaatgttttgtcctatcgtcagggtctctggaaatttgacacagagtgctataaaaagtatggaaaaatgtggggtatctcttccctgtttggaccacattacccttcatcatatgaagccttgggtggctcctgtgtgagactcttgctgtgtgtcacaccctaatgaactagaacctaaggttgctgtgtgtcgtacaactagggaacgtatgaaggtcaactccctgtgctggccatcacagatcccgacgtgatcagaacagtgctagtgaaagaatgttattctgtcttcacaaatcgaaggtctttaggcccagtgggatttatgaaaagtgccatctctttagctgaggatgaagaatggaagagaatacggtcattgctgtctccaaccttcaccagcggaaaactcaaggagatgttccccatcattgcccagtatggagatgtattggtgagaaacttgaggcgggaagcagagaaaggcaagcctgtcaccttgaaagacatctttggggcctacagcatggatgtgattactggcacatcatttggagtgaacatcgactctctcaacaatccacaagacccctttgtggagagcactaagaagttcctaaaatttggtttcttagatccattatttctctcaataatactctttccattccttaccccagtttttgaagcattaaatgtctctctgtttccaaaagataccataaattttttaagtaaatctgtaaacagaatgaagaaaagtcgcctcaacgacaaacaaaagcaccgactagatttccttcagctgatgattgactcccagaattcgaaagaaactgagtcccacaaagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaataaggcaccacctacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggttcagtaagaagaaggacagcatagatccttacatatacacaccctttggaactggacccagaaactgcattggcatgaggtttgctctcatgaacatgaaacttgctctaatcagagtccttcagaacttctccttcaaaccttgtaaagaaacacagatccccttgaaattagacacgcaaggacttcttcaaccagaaaaacccattgttctaaaggtggattcaagagatggaaccctaagtggagaatgagttattctaaggatttctactttggtcttcaagaaagctgtgccccagaacaccagagatttcaacttagtcaataaaaccttgaaataaagatgggcttaatctaaaaaaaaaaaaaa

SEQ ID NO: 45 - DNA, 437 bp, Homo sapiens
gcaggatttcaatgaccagcccacaaaagtatcctgtgtactactagttgaggggtggcccctaagtaagaaaccctaacatgtaactcttaggggtattatgtcattaactttttaaaaatctaccaacgtggaaccagattcagcaagaagaacaaggacaacatagatccttacatatacacaccctttggaagtggacccagaaactgcattggcatgaggtttgctctcatgaacatgaaacttgctctaatcagagtccttcagaacttctccttcaaaccttgtaaagaaacacaggttagtcaattttctataaaaataatgttgtattaataattcttttaactgagtggtctgtattttttaaaaagaatatgcttgtttaatcttttactaatttgttctctgggccaaagaatcaattaggccca

SEQ ID NO: 46 - DNA, 123 bp, artificial
  mutation
gcttaacgaatgctctactgtcatttctaaccataatctctttaaagagctcttttgtctttcaatatctcttccgtgtttggaccacattacccttcatcatatgaagccttgggtggctcc

SEQ ID NO: 47 - DNA, 23 bp, artificial
  Misc_RNA
ccagaaactgcattggcatgagg

SEQ ID NO: 48 - DNA, 23 bp, artificial
  Misc_RNA
agtggacccagaaactgcattgg

SEQ ID NO: 49 - DNA, 23 bp, artificial
  Misc_RNA
gtttgaaggagaagttctgaagg

SEQ ID NO: 50 - DNA, 23 bp, artificial
  Misc_RNA
gatgcagctggccctacgctggg

SEQ ID NO: 51 - DNA, 23 bp, artificial
  Misc_RNA
tctccatcacacccagcgtaggg

SEQ ID NO: 52 - DNA, 23 bp, artificial
  Misc_RNA
caatccacaagacccctttgtgg

SEQ ID NO: 53 - DNA, 23 bp, artificial
  Misc_RNA
actaagaagttcctaaaatttgg

SEQ ID NO: 54 - DNA, 23 bp, artificial
  Misc_RNA
cttcttagtgctctccacaaagg

SEQ ID NO: 55 - DNA, 40 bp, Homo sapiens
ccacgtatgtaccacccagcttggttacccttcccttctg

SEQ ID NO: 56 - DNA, 42 bp, Homo sapiens
ccacgtatgtaccacccagcttggaccacattacccttcatc

SEQ ID NO: 57 - DNA, 33 bp, Homo sapiens
gagctcttttgtctttcagtatctcttccctgt

SEQ ID NO: 58 - DNA, 33 bp, Homo sapiens
gagctcttttgtctttcaatatctcttccctgt

(19) United States
(12) Patent Application Publication
(10) Pub. No.: US 2020/0032269 A1
(43) Pub. Date: Jan. 30, 2020

(54) GENETICALLY MODIFIED CELL LINES FOR METABOLIC STUDIES

(71) Applicant: Hennepin Healthcare Research Institute, Minneapolis, MN (US)

(72) Inventors: Casey R. Dorr, Minneapolis, MN (US); Ajay K. Israni, Eden Prairie, MN (US)

(21) Appl. No.: 16/486,287

(22) PCT Filed: Feb. 16, 2018
(86) PCT No.: PCT/US2018/000037

§ 371 Date: Aug. 15, 2019

Related U.S. Application Data
(60) Provisional application No. 62/459,749, filed on Feb. 16, 2017.

(51) Int. Cl. C12N 15/113; C12Q 1/6876; G01N 33/50

(52) U.S. Cl. CPC C12N 15/1137; C12Q 2600/106; G01N 33/5008; C12Q 1/6876

(57) ABSTRACT

A cell line includes a genetic modification at a genetic locus that affects metabolism of a drug in a manner similar to a human subject having a genotype that affects metabolism of the drug. A method for making such a cell line generally includes identifying a locus and allelic variants at the locus in a human that affect metabolism of the drug, providing a parental cell line that comprises the locus that affects metabolism of the drug in the human subject, and introducing a genetic modification into the parental cell at the locus, thereby forming a variant cell line comprising an allelic variant at the locus having the genetic modification.

### CROSS-REFERENCE TO RELATED APPLICATION

This application claims priority to U.S. Provisional Patent Application No. 62/459,749, filed Feb. 16, 2017, which is incorporated herein by reference.

### GOVERNMENT FUNDING

This invention was made with government support under AI070119 and AI130409 awarded by the National Institutes of Health. The government has certain rights in the invention.

### SUMMARY

This disclosure describes, in one aspect, a method for making a cell line that metabolizes a drug in a manner similar to a human in which genotype of the cell affect metabolism of the drug. Generally, the method includes identifying in a human a locus and allelic variants at the locus that affect metabolism of the drug, providing a parental cell line that includes the locus that affects metabolism of the drug in the human, and introducing a genetic modification into the parental cell at the locus, thereby forming a variant cell line that includes an allelic variant at the locus having the genetic modification.

In some embodiments, the method further includes analyzing metabolism of the drug by the variant cell line compared to a human homozygous for the wild-type allele at the locus. In some of these embodiments, the method further includes identifying the variant cell line as a model cell line approximating metabolism of the drug by a human homozygous for the wild-type allele at the locus.

In some embodiments, the method further includes analyzing metabolism of the drug by the variant cell line compared to a human subject possessing at least one non-wild-type allele at the locus. In some of these embodiments, the method further includes identifying the cell line as a model cell line approximating metabolism of the drug by a human possessing at least one non-wild-type allele at the locus.

In some embodiments, the method further includes creating additional model cell lines. In some of these embodiments, an additional model cell line can include a different genetic modification into the parental cell line at the locus, thereby producing an additional variant cell line that differs from the first variant cell line. In some of these embodiments, the method can include analyzing the metabolism of one or more of the additional variant cell lines and, optionally, identifying an additional variant cell line as a model cell line approximating metabolism of the drug by a human possessing at least one non-wild-type allele at the locus.

In some embodiments, the method further includes an alternative process for creating additional model cell lines. In some of these embodiments, an additional model cell line can include. In these embodiments, the method includes introducing a second genetic modification into an already variant cell line at a second locus, thereby producing a second variant cell line that differs from the first variant cell line. In some of these embodiments, the method can include analyzing the metabolism of the variant cell line having the second genetic modification. In some of these embodiments, the method further includes identifying the variant cell line having the second genetic modification as a model cell line approximating metabolism of the drug by a human possessing at least one non-wild-type allele at the locus.

In some embodiments, the method further includes analyzing metabolism of the drug by the variant cell line compared to a human homozygous for the wild-type allele at the locus and adjusting the dose of the drug to a human patient. In some of these embodiments, adjusting the dose of drug to a human patient can include increasing the dose of the drug to a patient having at least one non-wild-type allele at the locus if the cell line metabolizes the drug more rapidly than a human homozygous for the wild-type allele at the locus. In other embodiments, adjusting the dose of drug to a human patient can include decreasing the dose of the drug to a patient having at least one non-wild-type allele at the locus if the cell line metabolizes the drug more slowly than a human homozygous for the wild-type allele at the locus.

In some embodiments, the parental cell can be an immortal cell line.

In some embodiments, the parental cell is, or is derived from, a human cell. In some of these embodiments, the human cell can be a liver cell, an intestinal cell, a kidney cell, stomach cell, colorectal cell, pancreatic cell, bile duct cell, urinary tract cell, or nervous system cell.

In some embodiments, the locus is a locus that influences Cytochrome P450 (CYP) activity. In some of these embodiments, the locus is CYP3A5, CYP3A4, CYP3A4, CYP3A43, CYP3A7, CYP3AP1, CYP2C9, CYP2C8, CYP2C19, CYP1A2, CYP2D6, CYP2E1, CYP2B6, CYP2A6, CYP2J2, POR, CYB5A, CYB5R1, CYB5R2, CYB5R3, CYB5R4, CYB5RL, CYB5D1, ABCB1 (MDR1), ABCC1 (MRP1), ABCC2 (MRP2), ABCG2, ABCE1/RNS4I, SLCO1B3, VDR, NR3C1 (GR), NR1I2 (PXR), NR1I3 (CAR), HNF4A, CEBPA, CEBPB, PPARA, FOXA2, NCOR1, or YY1.

In another aspect, this disclosure describes a method of providing personalized dosing of a drug to a patient whose genotype at a locus affects metabolism of the drug. Generally, the method includes determining the genotype of the patient at the locus; providing a model cell line having a genetic modification at the locus, the genetic modification causing the model cell line to approximate metabolism of the drug by the patient; culturing the cell line in the presence of the drug; evaluating metabolism of the drug by the cell line; and administering to the patient a dose of the drug based on metabolism of the drug by the cell line.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. A general process of developing a variant cell line associated with metabolism.

FIG. 2. A process of testing genetic variants associated with metabolism.

FIG. 3. Maps of CYP3A5 alleles in exemplary variant cell lines showing genetic modifications.

FIG. 4. DNA sequencing of exemplary CYP3A5 alleles in variant cell lines. (A) HuH-7 (CYP3A5 *3/*3; SEQ ID NO:34) compared to CYP3A5 *1/*3 sd; SEQ ID NO:55). (B) HuH-7 (CYP3A5 *3/*3; SEQ ID NO:34) compared to CYP3A5 *1/*1 dd; SEQ ID NO:56). (C) HuH-7 (CYP3A5 *3/*3; SEQ ID NO:57) compared to CYP3A5 *1/*3 pm; SEQ ID NO:58).

FIG. 5. Map of CYP3A5 genomic DNA showing the region of genetic modification performed in exemplary variant cell lines.

FIG. 6. Polymerase chain reaction (PCR) of genomic DNA at the CYP3A5 locus in exemplary variant cell lines.

FIG. 7. Region of variation among CYP3A5 mRNA variants. Additional nonvariant regions of the CYP3A5 mRNA are not shown.

FIG. 8. mRNA sequencing comparing nucleotide sequence of CYP3A5 at Exon 3B (SEQ ID NO:37) with corresponding regions in exemplary cell lines. (A) CYP3A5 *1/*3 sd (SEQ ID NO:38); (B) CYP3A5 *1*1 dd (SEQ ID NO:39); and (C) CYP3A5 *1/*3 pm (SEQ ID NO:40) cell lines.

FIG. 9. CYP3A5 mRNA splicing assay showing that exemplary variant cell lines express different mRNA than HuH-7 cells.

FIG. 10. Quantitative RT-PCR assay demonstrating the elevated mRNA expression in exemplary cell lines compared to HuH-7.

FIG. 11. A schematic diagram illustrating a metabolism assay with a drug as a substrate and its metabolic products as metabolites.

FIG. 12. A bar graph showing tacrolimus metabolism data by exemplary variant cell lines.

FIG. 13. A bar graph showing midazolam metabolism data by exemplary variant cell lines.

FIG. 14. A bar graph showing 1-OH midazolam production by exemplary variant cell lines data from an MDZ substrate.

FIG. 15. A bar graph illustrating 4-OH midazolam production by exemplary variant cell lines from a MDZ substrate.

FIG. 16. A schematic diagram comparing mechanisms of CYP3CIDE inhibition. CYP3A4 enzyme is inhibited with higher potency than CYP3A5 enzyme by CYP3CIDE.

FIG. 17. CYP3CIDE dose response curve in HuH-7 and CYP3A5 *1*1 dd cell lines.

FIG. 18. A bar graph showing data on the effect of CYP3C1DE on midazolam metabolism by exemplary variant cell lines.

FIG. 19. A bar graph showing data on the effect of CYP3CIDE on 1-OH midazolam production by exemplary variant cell lines.

FIG. 20. A bar graph showing data on the effect of CYP3CIDE on 4-OH midazolam production by exemplary variant cell lines.

FIG. 21. Nucleotide sequences of gRNAs for genetic modification of CYP3A5*3 locus. Variant nucleotide sequences in gRNA1-3 (SEQ ID NO:41) and gRNA 2-3 (SEQ ID NO:42) are underlined and in bold.

FIG. 22. Nucleotide sequence (SEQ ID NO:43) showing features of gRNAs for CRISPR modification of CYP3A5*3 locus. 1-3 gRNA PAM sequence: a three-base proto-spacer adjacent motif (PAM) sequence for Cas9 recognition; gRNA loci: underlined; intron sequences: lower case; Exon 3B: upper case.

FIG. 23. Nucleotide sequence of CYP3A5 mRNA (SEQ ID NO:44; GenBank accession no. BC025176.1). Underlined: primer locations; bold: Exon 3B sequence present in CYP3A5*3 but absent in CYP3A5*1. Italics and non-italicized text represent alternating exons.

FIG. 24. Surveyor assay for CYP3A5*3 locus in HuH-7 cell line to assess the effect of genetic modification.

FIG. 25 Nucleotide sequence surrounding CYP3A4*3 locus (SEQ ID NO:45). lower case: intron sequences; upper case: exon sequence; underlined: primers; bold: CYP3A4 *3 locus.

FIG. 26. Map of all the three gRNAs designed targeting CYP3A4*3 locus.

FIG. 27. Surveyor assay for CYP3A4*3 locus in HuH-7 cell line to assess the effect of genetic modification with three different guide RNAs targeting the CYP3A4*3 locus.

FIG. 28. Flow chart of a process for determining the association of genetic variants with a drug metabolism or metabolic products.

### DETAILED DESCRIPTION OF ILLUSTRATIVE EMBODIMENTS

The disclosure describes compositions and methods for drug and pharmaceutical testing, and more particularly to pharmaceutical dosing based on genetic variations in populations. This disclosure also relates to genetic modification of cell lines and testing the effects of those genetic variants on drug metabolism.

Broadly, embodiments of the present invention provide a composition of matter and a process to develop a composition of matter that is useful for determining an association of genetic variants with drug metabolism or a metabolic product. Generally, the process involves genetically modifying a cell line to express genetic variants. The compositions of matter that the process produces are new, genetically distinct cell lines. Three exemplary cell lines produced using the process described herein are the human liver cell lines deposited at American Type Culture Collection (ATCC) under the Budapest Treaty (with ATCC Accession No): CYP3A5 *1/*3 sd (PTA-123710), CYP3A5 *1/*1 dd (PTA-124693), and CYP3A5 *1/*3 pm (PTA-124694).

The method described herein can be used to develop genetically modified cell lines that vary, with respect to its parental cell line, in drug metabolism the genes associated with drug metabolism. For example, CRISPR/Cas9 technology can introduce the *1 allele into the CYP3A5 gene in human liver cells that originally possessed the *3 allele. The exemplary modified cells were characterized by DNA sequencing, mRNA sequencing, polymerase chain reaction (PCR), RNA splicing assay, Reverse Transcriptase-PCR (RT-PCR), western blot, and drug metabolism assays using tacrolimus (Tac) or midazolam (MDZ) as a substrate, which were consistent with the effects of allele changes seen in human subjects.

Human liver cell lines can be engineered using CRISPR/Cas9, TALEN, zinc fingers, or other methods to engineer cells to express variants for drug metabolism analysis. This concept can be used to study an assortment of genetic variants and a variety of drugs leading to various applications of this technology for pharmacogenomics, pharmacokinetic, and other studies. Thus, while described below in the context of an exemplary embodiment in which cells are genetically modified using CRISPR/Cas9 technology, the methods described herein can be performed, and cell lines may be produced, using other molecular biology techniques suitable for genetically modifying cells.

Use of liver cells from animals is less clinically relevant than using human cells. However, primary human hepatocytes are not an optimal option because they require harvesting of the liver cells from humans, are not immortalized, and there is a great deal of genetic and environmental variability between liver cells acquired in this manner. However, primary cells can be immortalized and can be efficient as a parental cell line for this process. While liver microsomes can be effective to study drug metabolism, they cannot be genetically engineered to study genetic variants such as those found in minority patient populations. However, microsomes created from cells genetically modified using the methods described herein could be useful for testing genetic association with metabolism.

Many patients are treated with suboptimal doses of drugs, which can be improved by using the genotype of the patient to determine the appropriate dose. Alternatively, some patients are overdosed with drugs which can lead to toxicity. This disclosure describes methods in which genetically-modified cells can provide an efficient and well-controlled model for determining the role of genotype in drug metabolism.

This disclosure describes three exemplary cell lines, each of which expresses a different CYP3A5 allele. CYP3A5 is a human gene that encodes cytochrome P450 (CYP) 3A5, a monooxygenase that catalyzes many reactions involved in drug metabolism. CYP3A5 is expressed, among other places, in human liver cells. The CYP3A5 *1 allele is a variant prevalent in African Americans populations and leads to rapid metabolism of midazolam (MDZ), tacrolimus (Tac), and other drugs. The CYP3A5 *3 allele is a variant prevalent in people of Caucasian descent and leads to low rates of metabolism of drugs such as Tac, used in solid organ transplantation, and MDZ, which is used as a sedative or anesthetic. The CYP3A5 *1 allele and the CYP3A5 *3 allele are not limited to any single population, however. Thus, the genotype of a patient is an important factor in considering the dose of a drug that is metabolized through the CYP pathway.

As one relevant clinical example, patients carrying a CYP3A5 *1 allele are often underdosed with tacrolimus following organ transplantation. Underdosing may explain, at least in part, the increased allograft rejection and poor clinical outcomes witnessed in African Americans, in which the CYP3A5 *1 allele is more prevalent, because they are often dosed the same as the general population. Patients carrying a CYP3A5 *1 allele may require a higher dose of Tac to achieve therapeutic target drug levels. But, the CYP3A5 *1 allele is not universally present in all African Americans. Thus, merely dosing based on race can be unreliable since some patients may be assumed to carry the CYP3A5 *1 allele but do not. If these patients receive a higher dose, they may, in fact, be overdosed, which can lead to toxicity. Thus, the methods described herein allow one to determine a patient's genotype and then to administer a dose appropriate to the individual patient's genotype rather than administering the dose based on the general population or race.

Genotype-based dosing is also important for other genetic variants associated with human drug metabolism. Thus, the methods described herein allow one to determine the relationship between genotype and drug dose in a cell culture prior to trial and error testing in humans. By determining the effect of genotype on metabolism, one can more accurately administer personalized medicine while minimizing clinical trial time.

HuH-7 was used as the parental cell line for the exemplary variant cell lines constructed as described in the Examples. The HuH-7 cell line (Nakabayashi et al., 1985, Cancer Res 45(12 Pt 1):6379-6383) can convert MDZ in cell culture to its metabolites, hydryoxylated 1-OH MDZ and 4-OH MDZ, but is not very efficient because HuH-7 is a homozygous genotype for the non-functional CYP3A5 *3/*3 alleles.

While described herein in the context of an exemplary embodiment in which the parental cell line was HuH-7, the methods described herein can be practiced using any suitable cell line. Exemplary alternative parental cell lines include, but are not limited to, HepG2, HUH6, Hep3B, LS-180, Caco-2, HLE, HLF, SKHEP, CYP3A5 *1/*3 sd, CYP3A5 *1/*1 dd, or CYP3A5 *1/*3 pm.

Also, while described above in the context of an exemplary embodiment in which the CYP3A5 locus is the target of the genetic modifications, the methods described herein can be used to create cell lines that vary at any locus of interest. Exemplary alternative loci involved in drug metabolism include, for example, CYP3A4, CYP3A43, CYP3A7, CYP3AP1, CYP2C9, CYP2C8, CYP2C19, CYP1A2, CYP2D6, CYP2E1, CYP2B6, CYP2A6, CYP2J2, POR, CYB5A, CYB5R1, CYB5R2, CYB5R3, CYB5R4, CYB5RL, CYB5D1, ABCB1 (MDR1), ABCC1 (MRP1), ABCC2 (MRP2), ABCG2, ABCE1/RNS4, SLCO1B3, VDR, NR3C1 (GR), NR1I2 (PXR), NR1I3 (CAR), HNF4A, CEBPA, CEBPB, PPARA, FOXA2, NCOR1, or YY1. Exemplary loci include loci in any member of the CYP gene families CYP1, CYP2, CYP3, CYP4, CYP5, CYP7, CYP8, CYP11, CYP17, CYP19, CYP20, CYP21, CYP24, CYP26, CYP27, CYP39, CYP46, or CYP51.

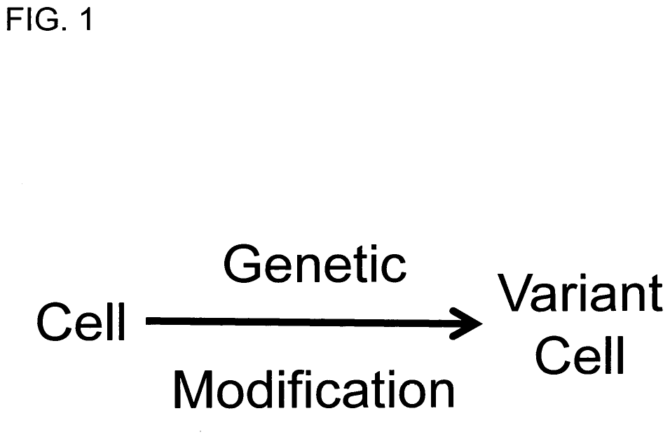

FIG. 1 shows a general process of developing a variant cell line associated with metabolism. According to the process, a genetic modification is applied to a cell line to produce a variant cell line.

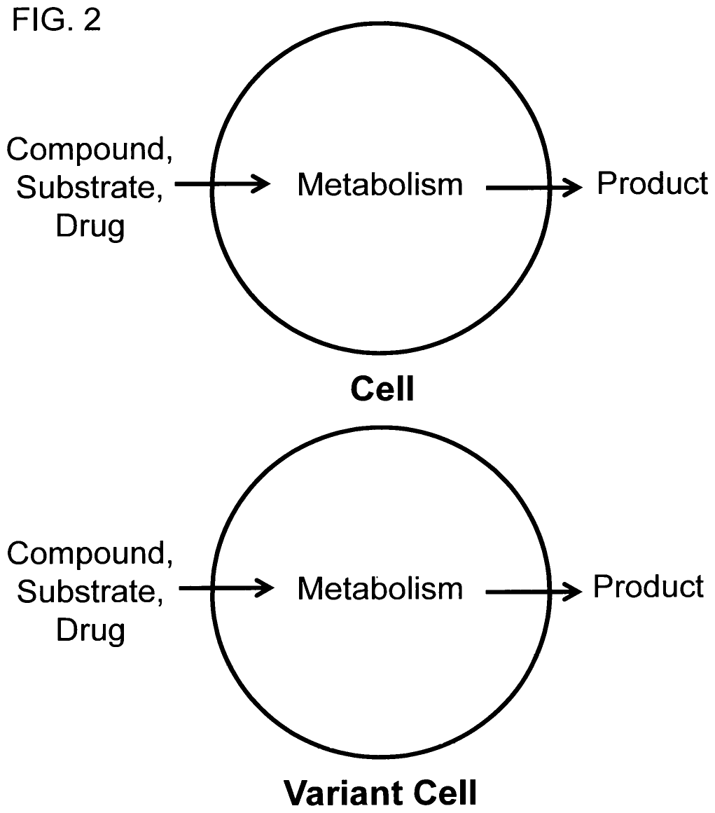

FIG. 2 illustrates a process of testing genetic variants associated with a cell metabolic process. According to this process, a cell is exposed to a compound, a drug, or a substrate, and the cell's metabolic process produces a metabolic product. Similarly, the compound, drug, or substrate is applied to the variant cells, which produces a metabolic product. The metabolic products are then assayed to determine the genetic variant's role in metabolism.

The exemplary new cell lines described herein have been engineered using CRISPR technology and characterized through DNA sequencing, mRNA sequencing, PCR, RNA splicing assay, RT-PCR, western blot, and drug metabolism assays using tacrolimus or midazolam as a substrate. Three exemplary cell lines are expressly described herein. Each exemplary cell line is a different human hepatocyte cell line, characterized as described below.

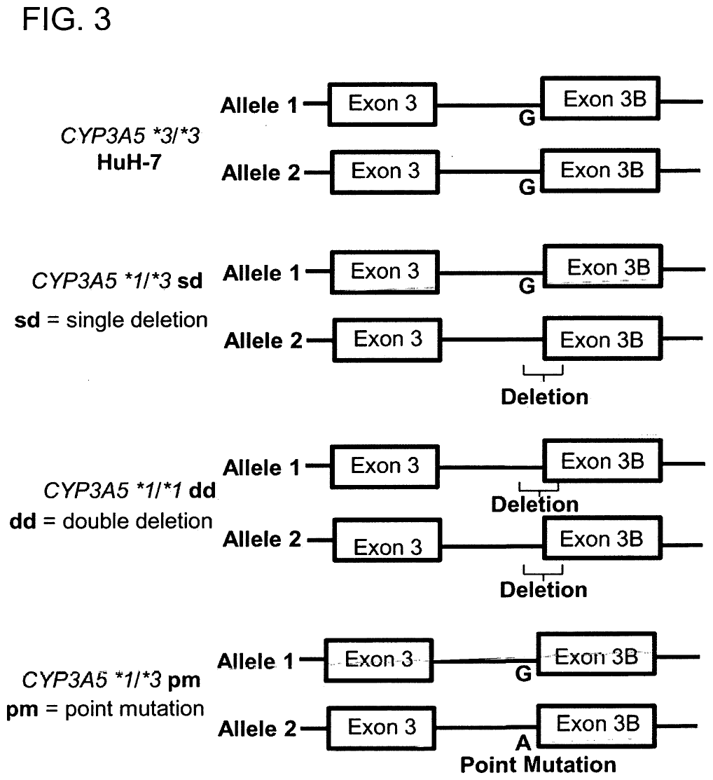

FIG. 3 illustrates the genetic modification in the exemplary cell lines. The CYP3A5 *1/*3 sd cell line is heterozygous at the CYP3A5 locus, having one CYP3A5 *3 allele and a second allele having a deletion at the mRNA splice junction upstream of CYP3A5 Exon 3B. The CYP3A5 *1/*1 dd cell line is homozygous, having a deletion at the mRNA splice junction upstream of CYP3A5 Exon 3B on both alleles. The CYP3A5 *1/*3 pm cell line is heterozygous, having one CYP3A5 *3 allele and a second allele having a guanine (G) to an adenine (A) substitution at the mRNA splice junction upstream of CYP3A5 Exon 3B.

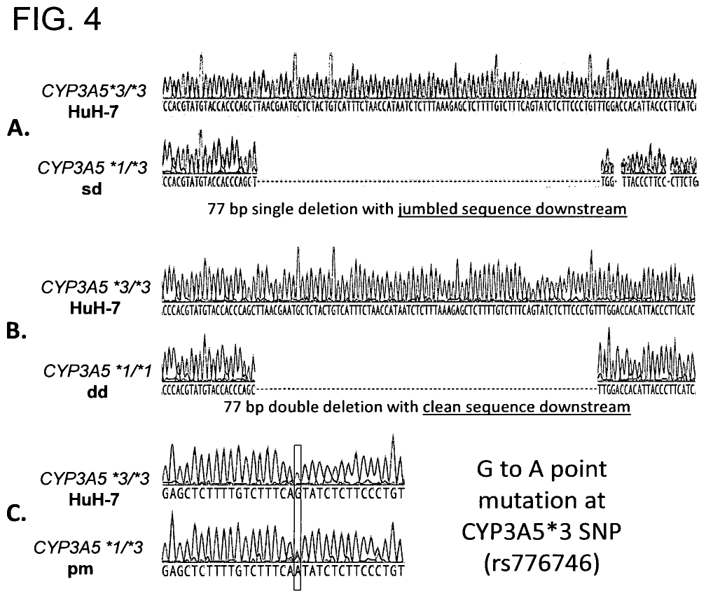

FIG. 4 shows the nucleotide sequences of the variant CYP3A5 alleles compared to the *3/*3 genotype. The CYP3A5 *1/*3 sd cell line has a heterozygous deletion and the sequence is a mixture between the two alleles with one allele being frameshifted. The sequence alignment of this cell line with HuH-7 cell line shows the deletion and the jumbled sequence downstream of deletion indicating heterozygosity (FIG. 4A). The CYP3A5 *1/*1 dd cell line has a deletion of 77 base pairs surrounding the CYP3A5 Exon 3B mRNA splice junction. The sequence alignment of this cell line with HuH-7 cell line shows this deletion and clean sequence downstream of the deletion (FIG. 4B). The CYP3A5 *1/*3 pm cell line has a guanine (G) to an adenine (A) substitution, compared to the corresponding nucleotide sequence in the HuH-7 parental cell line, at the mRNA splice junction upstream of CYP3A5 Exon 3B that is present on one of the alleles in CYP3A5 *1/*3 pm cell line (FIG. 4C). The sequence peak G is also seen together with sequence peak A indicating the presence of both *1 and *3 alleles.

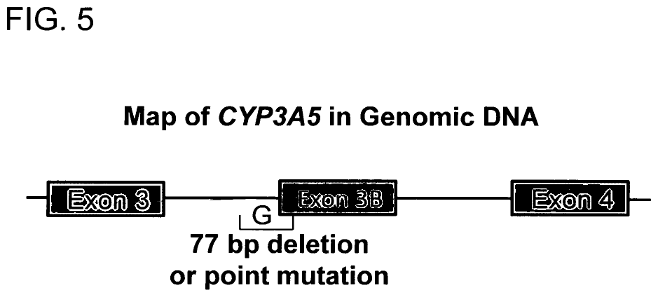

FIG. 5 shows the map of CYP3A5 genomic DNA spanning Exon 3, Exon 3B, and Exon 4 and the genetic modifications seen in the exemplary cell lines.

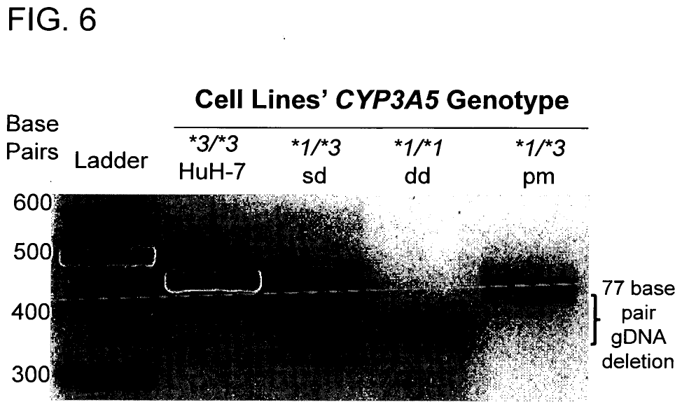

FIG. 6 shows 2% agarose gel electrophoresis of PCR products spanning the CYP3A5 *3 locus (rs776746) of genomic DNA from the parental HuH-7 cell line and the three exemplary variant cell lines. CYP3A5 *1/*3 sd had two different alleles, one allele being the same as the HuH-7 reference and one allele being 77 base pairs shorter than the reference. CYP3A5 *1/*1 dd showed no reference allele and thus deletion of the Exon 3B in both alleles. CYP3A5 *1/*3 pm has one PCR product the same size as the reference which is expected in a point mutant. The 77-bp deletion, seen in CYP3A5 *1/*3 sd and CYP3A5 *1/*1 dd, of the splice junction of CYP3A5 Exon 3B, prevents Exon 3B from being incorporated into the CYP3A5 mRNA, and thus is non-functional. CYP3A5 *1/*3 pm does not have a deletion of the DNA as seen in the figure. CYP3A5 *1/*3 pm is a single base change at the *3 (rs776746) SNP locus from a guanine (G) to an adenine (A) making it a CYP3A5 *1 variant. Thus, CYP3A5 *1/*3 sd CYP3A5 *1/*1 1dd, and CYP3A5 *1/*3 pm are genetically distinct from each other.

To characterize the CYP3A5 expression in the variant cell lines, mRNA splicing was assessed and the mRNA was sequenced to determine the expressed mRNA variant. The mRNA was isolated from the cells and converted to cDNA with an oligo-dT primer.

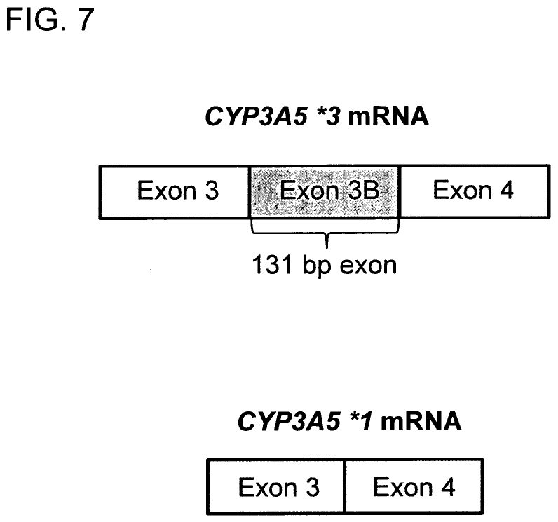

FIG. 7 shows the map of CYP3A5 Exon 3, Exon 3B, and Exon 4, showing the difference between the CYP3A5 *1 and CYP3A5 *3 mRNA variants. Specifically, the non-functional CYP3A5 *3 allele includes the 131 bp Exon 3B, while the CYP3A5 *1 allele does not.

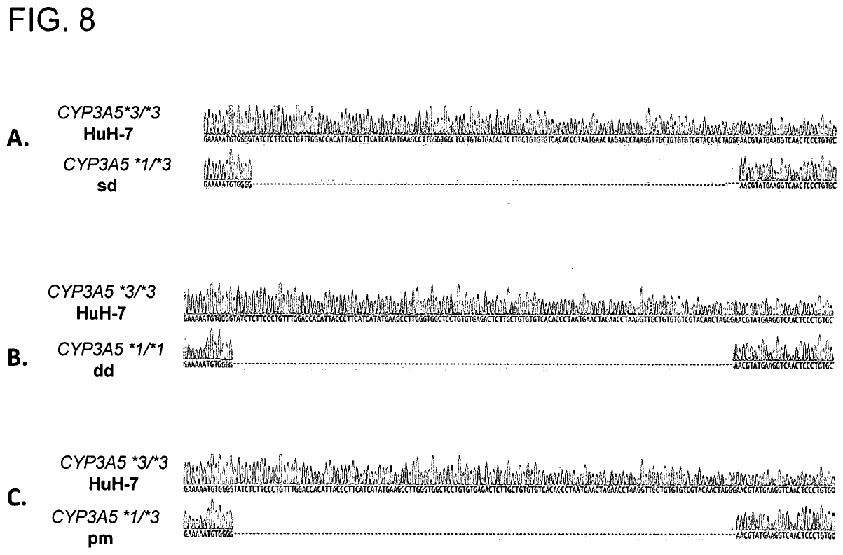

FIG. 8 is sequence chromatograms of cDNA from CYP3A5 *3 locus mRNA in cell lines. Total RNA was isolated from the cell lines, reverse transcribed with oligo dT primer and the CYP3A5 mRNA cDNA was sequenced with primers CYP3A5 cDNA ex2F (5′-GTCACAATCCCTGTGACCTGAT-3; SEQ ID NO:20) or CYP3A5 cDNA ex5R (5′-TTGGAGACAGCAATGACCGT-3′; SEQ ID NO:21)) that flank the 131-base-pair exon 3B. All cell sequences were aligned to HuH-7 reference control that was CYP3A5 *3/*3 genotype. The sequence alignment of CYP3A5 *1/*3 sd cell line shows the absence of Exon 3B in this cell line. It is possible that the *1 CYP3A5 was seen in this sequence because previous reports show that CYP3A5 *3 mRNA is targeted for non-sense mediated decay and would be more difficult to identify in a heterozygote. The sequence alignment of CYP3A5 *1/*1 dd and CYP3A5 *1/*3 pm also shows the absence of Exon 3B in these cell lines. Since CYP3A5 *1/*1 dd is homozygous for CYP3A5*1/*1, there is a clean sequence after the deletion.

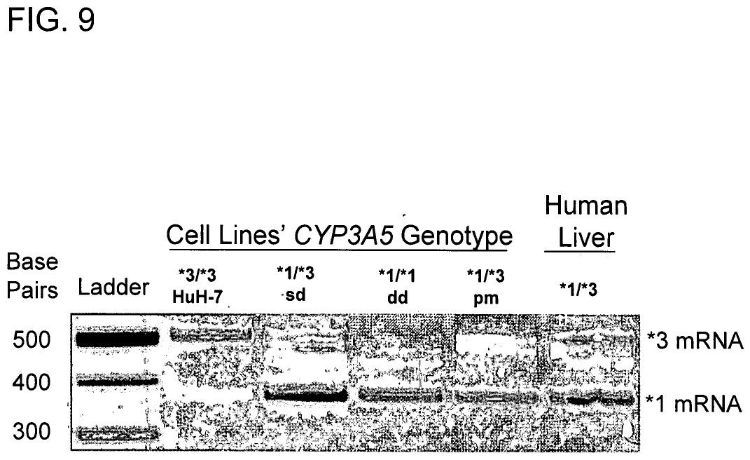

FIG. 9 shows RT-PCR products, with cDNA as template from oligo-dT primed cell line RNA. The gene specific primers CYP3A5 cDNA ex2F (5′-GTCACAATCCCTGTGACCTGAT-3 (SEQ ID NO:20); CYP3A5 cDNA ex5R (5′-TTGGAGACAGCAATGACCGT-3′ (SEQ ID NO:21)) were used to amplify the cDNA and electrophoresed in a 2% agarose gel. Genotyped human liver from a CYP3A5 *1/*3 patient cDNA was used as control. The CYP3A5 *1/*1 dd cell line showed the homozygous expression of CYP3A5 *1 mRNA and complete absence of *3 mRNA which has the additional 131 bp Exon 3B. CYP3A5 *1/*3 sd and CYP3A5 *1/*3 pm cell lines had heterozygous expression of both CYP3A5 *1 and CYP3A5 *3 mRNA splice variants, as compared with the heterozygous human liver cDNA. FIG. 9 also shows that CYP3A5 *1 mRNA can be expressed by either deleting the splice junction, as in CYP3A5 *1/*3 sd and CYP3A5 *1/*1 dd cell lines, or by point mutation by converting the G to an A at the mRNA splice junction as in CYP3A5 *1/*3 pm cell line. Both methods prevent the splicing of Exon 3B into the mature mRNA.

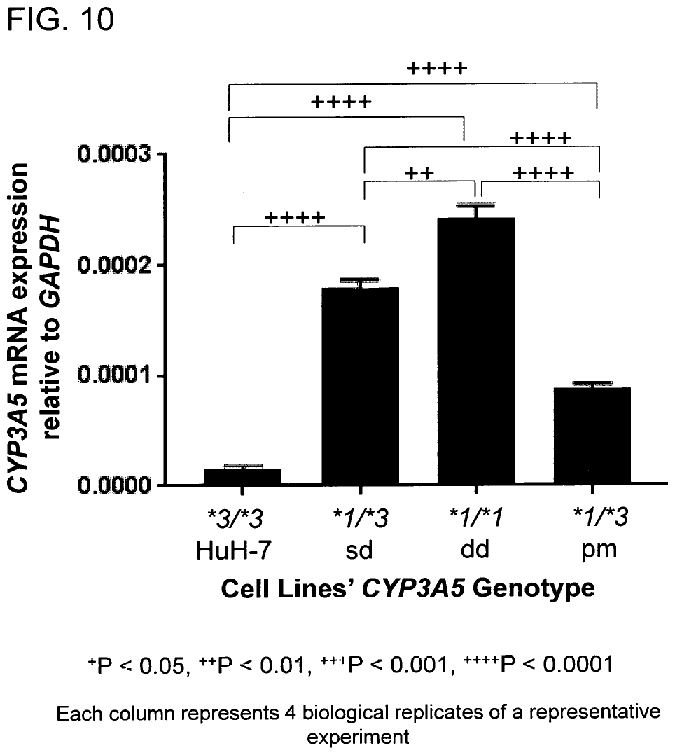

FIG. 10 shows that the mRNA expression in all three exemplary cell lines is significantly higher when compared with HuH-7 parental cell line. Total RNA was extracted from all three exemplary cell lines after exposure to MDZ and Tac, reverse transcribed and estimated using SYBR-Green assay based quantitative RT-PCR method. The primers used were same as RNA splicing assay. GAPDH primers were used as the reference control. FIG. 10 shows expression of CYP3A5 in cell lines relative to GAPDH. P-values comparing CYP3A5 mRNA expression levels between cell lines are from a paired two-sample t test show the exemplary cell lines have significantly higher levels of CYP3A5 mRNA than the HuH-7 parental cell line.

Aspects of the present invention include the process to develop cell lines associated with compound metabolism, the metabolism assay, and the characterization of the cell lines. Once the cell lines are engineered, the cell lines are then characterized using molecular, chemical, and/or cellular biological methods such as, for example, the characterization methods described herein. The metabolic activity of the variant cell lines can be assessed in cell culture. The functional assays are used to determine the metabolic activity of the cell lines. The metabolism assays are used to determine the function of the genetic variants.

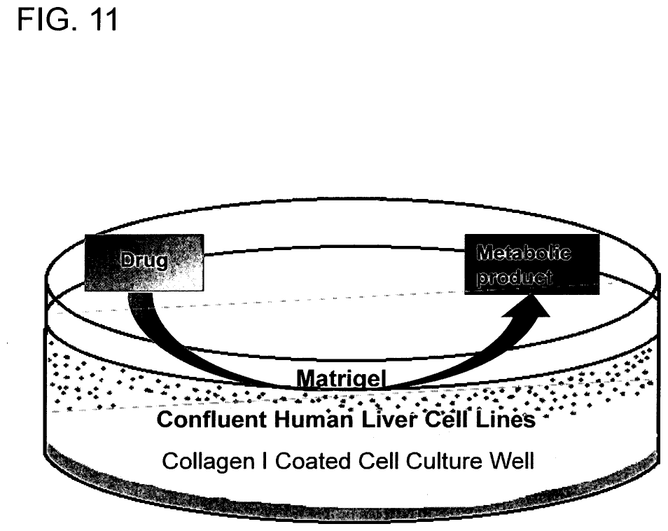

To further characterize the cell lines, metabolism assays were performed to determine the effect of each genetic variant on metabolism. FIG. 11 illustrates a generalized representative assay. In this exemplary assay, a cell line is plated on a collagen I-coated plate (or in a well of a multi-well plate, etc.), layered with an extracellular-matrix-like hydrogel (e.g., MATRIGEL, Corning Life Sciences, Tewksbury, Mass.), induced with a suitable inducer (e.g., rifampin and phenytoin), then treated with the substrate MDZ. The MDZ substrate is converted by the metabolic processes of the cell line to 1-OH MDZ or 4-OH MDZ, which can be measured using, for example high performance liquid chromatography (HPLC). The assay can be altered depending on the substrate, cell line, genetic variant, and any other experimental conditions.

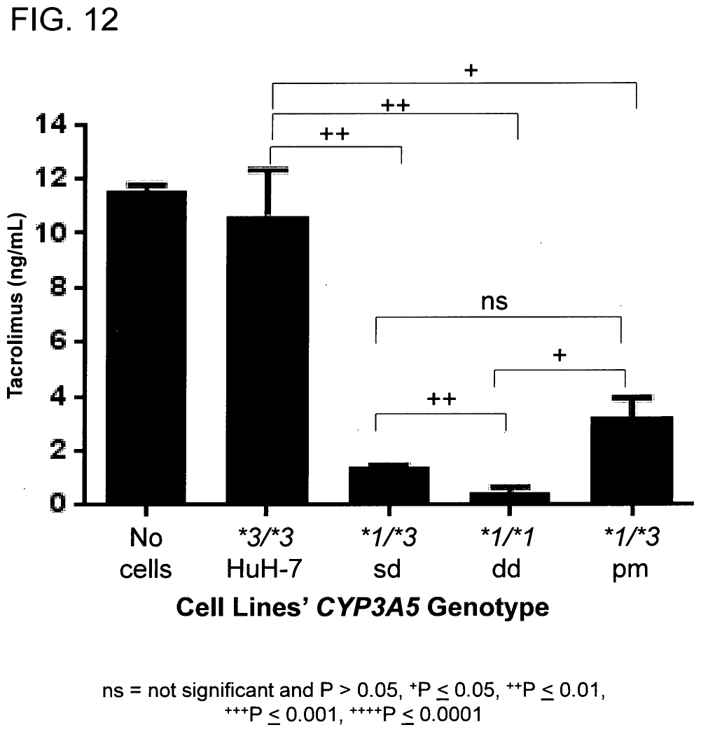

FIG. 12 shows data comparing metabolic Tac disappearance by each of the various cell lines. In this metabolism assay, all three exemplary cell lines and HuH-7 parental cell line were grown to confluency and then layered with MATRIGEL (Corning Life Sciences, Tewksbury, Mass.). The cells were then induced with rifampicin and phenytoin for three days and treated with substrate Tac overnight. Cell culture media were collected after treatment and assayed for Tac disappearance using liquid chromatography-mass spectrometry. The HuH-7 CYP3A5 *3/*3 parental cell line had higher level of Tac. FIG. 12 also shows exemplary cell lines metabolized Tac more efficiently than the parental HuH-7 cell line. CYP3A5 *1/*3 sd and CYP3A5 *1/*1 pm cell lines acted similarly in clearing Tac. CYP3A5 *1/*1 dd cell line exhibited the most Tac disappearance. Each column in the figure represents five biological replicates of a representative experiment and shows the disappearance of Tac caused by the cells' metabolism.

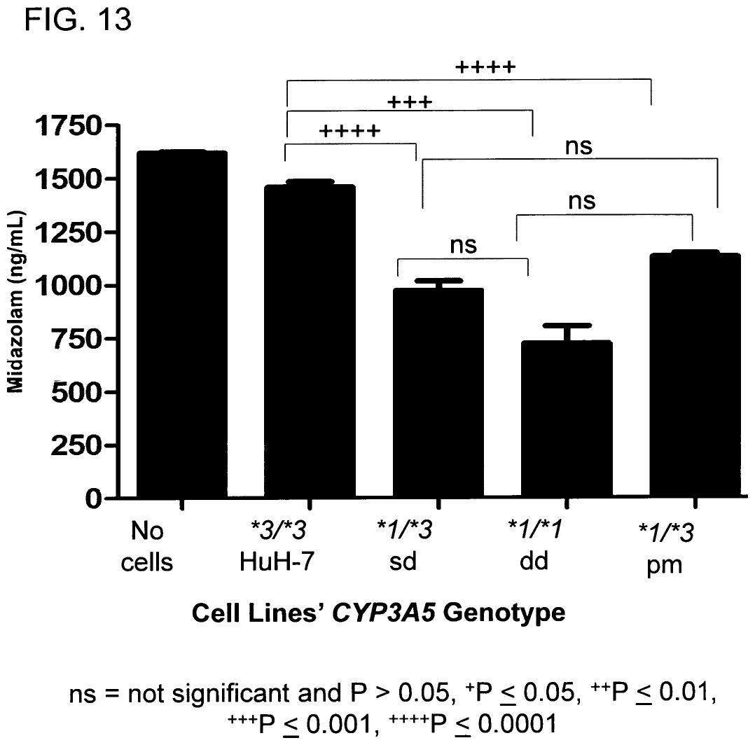

FIG. 13 shows data comparing metabolic MDZ disappearance by each of the various cell lines. The metabolism assay was performed same way as the Tac assay except the substrate used was MDZ. The exemplary CYP3A5 *1 expressing variant cell lines metabolized MDZ more efficiently than the parental HuH-7 cell line. CYP3A5 *1/*3 sd and CYP3A5 *1/*1 pm cell lines acted similarly in clearing MDZ. CYP3A5 *1/*1 dd cell line exhibited the most MDZ disappearance. Each column in FIG. 13 represents five biological replicates of a representative experiment and shows the disappearance of Tac caused by the cells' metabolism.

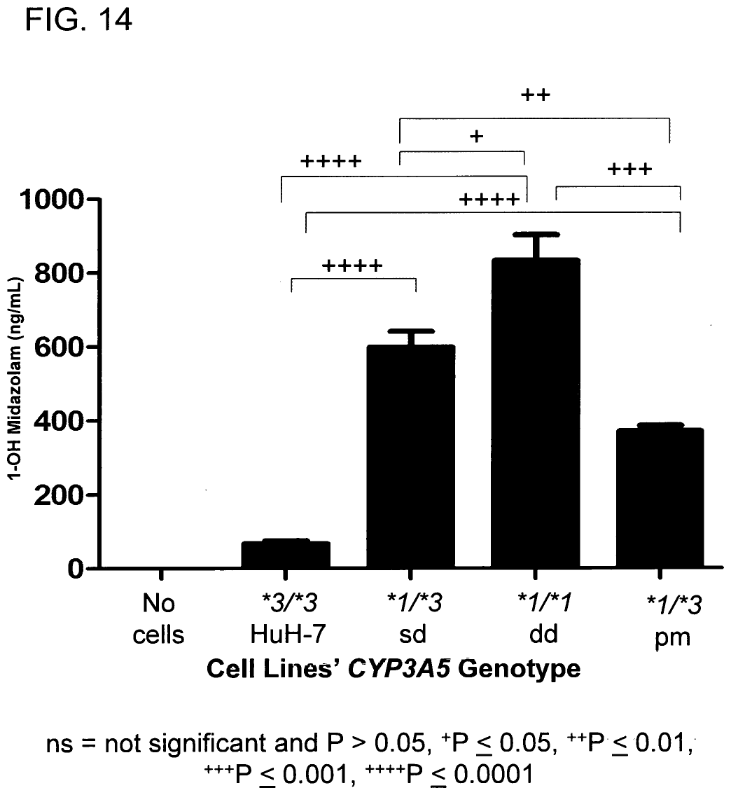

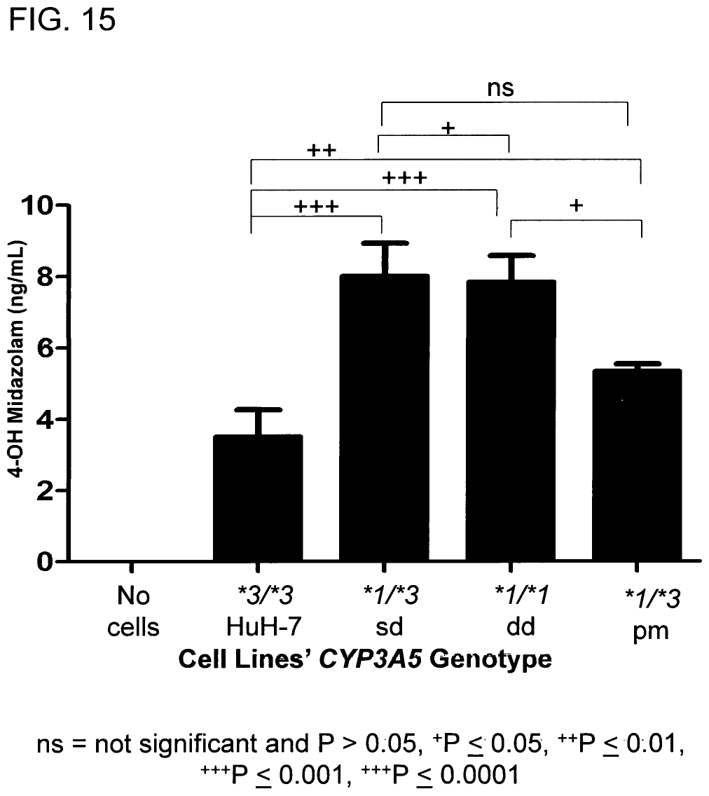

FIG. 14 shows the appearance of the metabolite 1-OH MDZ. The MDZ metabolism assay was performed and the product 1-OH MDZ was measured. CYP3A5 *1/*1 dd produced more 1-OH MDZ than the other cell lines. Similarly, CYP3A5 *1/*3 sd and CYP3A5 *1/*1 pm produced more 1-OH MDZ than does the HuH-7 parental cell line. Likewise, FIG. 15 shows production of the 4-OH MDZ metabolite by each of the cell lines. These data show that each of the genetically-modified cell lines is more active at MDZ metabolism than the parental HuH-7 cell line.

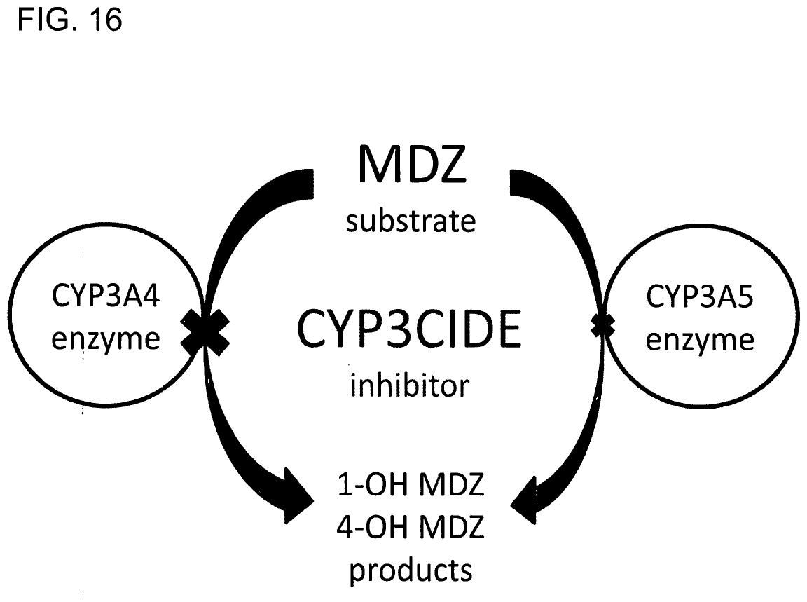

CYP3CIDE is a selective inhibitor of CYP3A4 enzymatic function and was used to differentiate between CYP3A4-dependent and CYP3A5-dependent metabolism. FIG. 16 is a diagram showing the relative inhibitory function of CYP3CIDE. CYP3CIDE has a mild inhibitory effect on CYP3A5 but is a strong inhibitor of CYP3A4. The parental HuH-7 cell line does not have functional CYP3A5. Each genetically-modified cell line was treated with CYP3CIDE to determine whether any of the genetic modifications in the genetically-modified cell lines produced CYP3A5 enzymatic activity.

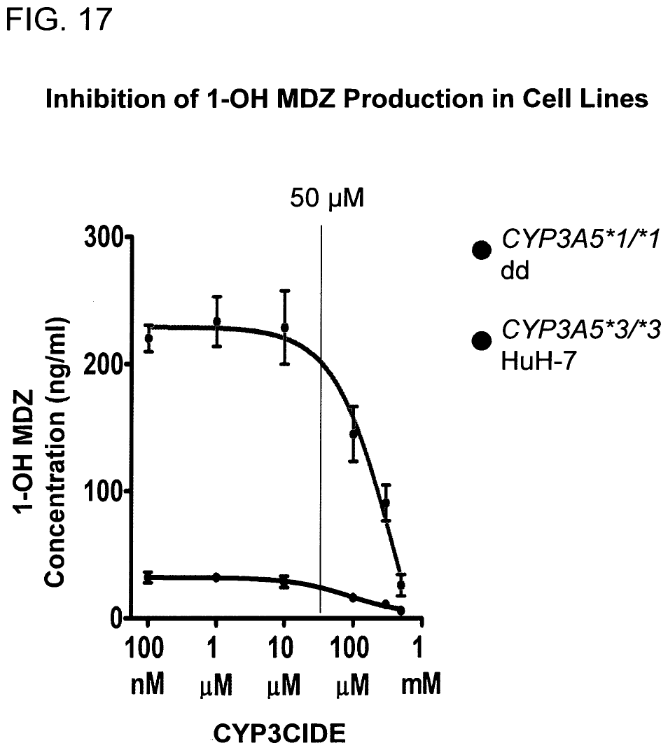

FIG. 17 shows the result of dose response performed to determine optimal concentration of CYP3CIDE to use in cell culture. The experiment was performed with HuH-7 and CYP3A5 *1/*1 dd cell lines where MDZ was used as substrate and between 100 nM to 1 mM concertation of CYP3CIDE was added. The dose response curve was drawn using the measured concertation of 1-OH MDZ product as metabolite. The optimal concentration chosen with this analysis was 50 μM of MDZ.

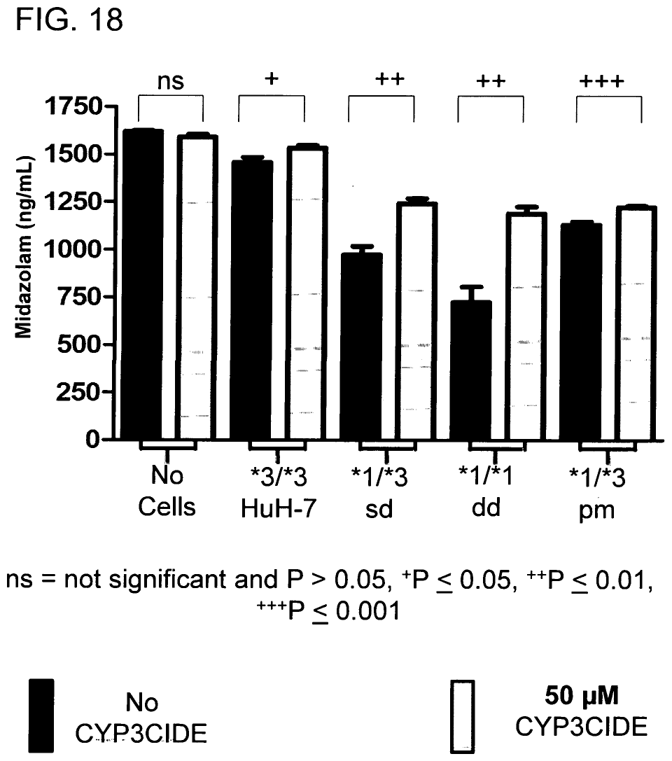

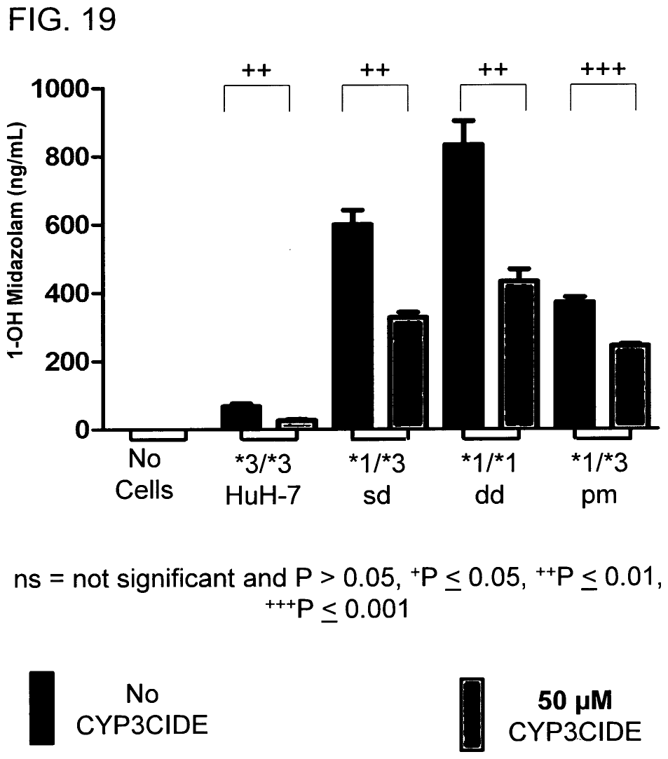

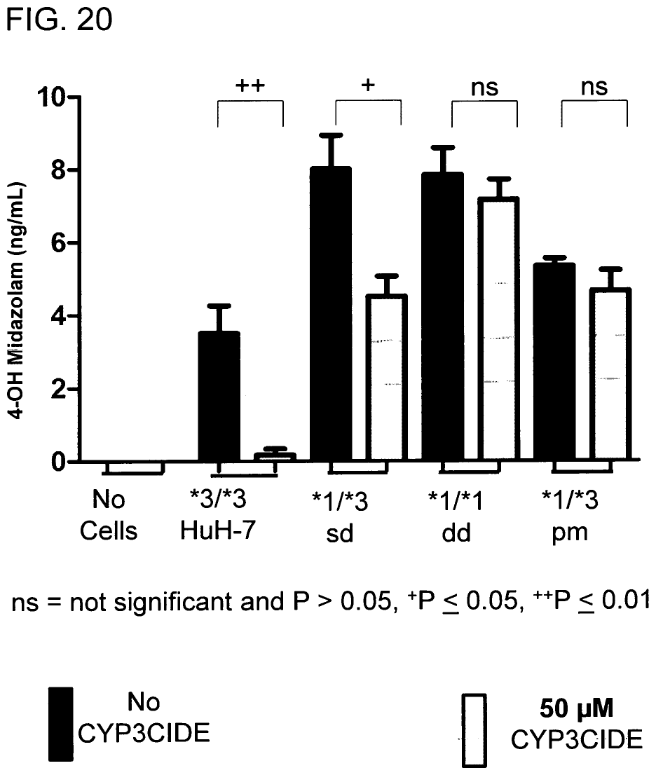

FIG. 18 is a bar graph showing that 50 μM CYP3CIDE inhibits the metabolism of MDZ in cell culture. When CYP3CIDE is present, there is more MDZ in the cell cultures. Similarly, FIG. 19 shows that 50 μM of CYP3CIDE inhibited the production of 1-OH MDZ from the cells. However, while the 1-OH MDZ production is nearly completely inhibited by CYP3CIDE in HuH-7 cells, the genetically-modified cell lines still produced a detectable level of 1-OH MDZ. FIG. 20 shows that production of 4-OH MDZ is inhibited by 50 μM CYP3CIDE in HuH-7 cells, while the genetically-modified cell lines still produce 4-OH MDZ. CYP3CIDE inhibited MDZ metabolism in HuH-7 cells more completely than in CYP3A5 *1/*3 sd, CYP3A5 *1/*1 dd, or CYP3A5 *1/*1 pm cell lines because the genetically-modified cell lines have a functioning CYP3A5 (*1 allele) while HuH-7 (*3 allele) does not. Thus, the exemplary cell lines have different MDZ metabolism and CYP3A function than the parental HuH-7 cells.

To further assess the difference in the exemplary genetically-modified cell lines and HuH-7 cell line in presence of CYP3CIDE, the metabolism data were analyzed using a two-tailed, paired, T-test. Table 1 compares the metabolism of MDZ with or without CYP3CIDE along with the p-values of the comparison. Table 2 presents a similar analysis of the appearance of the 1-OH MDZ product, while Table 3 presents a similar analysis of the appearance of the 4-OH product. These data show that CYP3CIDE influences MDZ metabolism in each of the cell lines. Table 4 presents data showing pair-wise comparisons between cell lines. All three exemplary genetically-modified cell lines metabolize Tac and MDZ more actively than the parental HuH-7 cell line.

TABLE 1

| | Midazolam | | |
|---|---|---|---|
| | (ng/mL)^{#} | | |
| | No | With | |
| Cell Line | CYP3CIDE\hat{ } | CYP3CIDE\hat{ } | P-Value^{@} |
| No Cells | 1590 ± 27 | 1620 ± 8.8 | 0.25874540 |
| HuH-7 | 1458 ± 62 | 1534 ± 32 | 0.04472352 |
| CYP3A5 *3/*3 | | | |
| CYP3A5 *1/*3 sd | 971 ± 110 | 1241 ± 64 | 0.003327 |
| CYP3A5 *1/*1 dd | 724 ± 200 | 1189 ± 90 | 0.00330988 |
| CYP3A5 *1/*3 pm | 1130 ± 45 | 1224 ± 18 | 0.00130289 |

^{#}Values are in ng/ml and the starting concentration was 5 μM (approximately 1629 ng/ml) MDZ.
\hat{ }Data represent the average of six samples of a representative experiment with standard deviation.
^{@}p-values were derived using a T-test.

TABLE 2

| | 1-OH Midazolam | | |
|---|---|---|---|
| | (ng/mL)^{#} | | |
| | No | With | |
| Cell Line | CYP3CIDE\hat{ } | CYP3CIDE\hat{ } | P-Value^{@} |
| No Cells | 0 ± 0 | 0 ± 0 | — |
| HuH-7 | 67 ± 22 | 26 ± 8.2 | 0.0142868 |
| CYP3A5 *3/*3 | | | |
| CYP3A5 *1/*3 sd | 599 ± 103 | 327 ± 36 | 0.002136 |
| CYP3A5 *1/*1 dd | 833 ± 172 | 432 ± 88 | 0.0040919 |
| CYP3A5 *1/*3 pm | 371 ± 37 | 243 ± 13 | 0.00075781 |

^{#}Values are in ng/ml and the starting concentration was 5 μM (approximately 1629 ng/ml) MDZ.
\hat{ }Data represent the average of six samples of a representative experiment with standard deviation.
^{@}p-values were derived using a T-test.

TABLE 3

| | 4-OH Midazolam | | |
|---|---|---|---|
| | (ng/mL)^{#} | | |
| | No | With | |
| Cell Line | CYP3CIDE\hat{ } | CYP3CIDE\hat{ } | P-Value^{@} |
| No Cells | 0 ± 0 | 0 ± 0 | — |
| HuH-7 | 3 ± 1.9 | 0 ± 0.5 | 0.00757727 |
| CYP3A5 *3/*3 | | | |
| CYP3A5 *1/*3 sd | 8 ± 2.2 | 5 ± 1.3 | 0.00261 |
| CYP3A5 *1/*1 dd | 8 ± 1.6 | 7 ± 1.4 | 0.0050069 |
| CYP3A5 *1/*3 pm | 5 ± 0.45 | 5 ± 1.4 | 0.33345497 |

^{#}Values are in ng/ml and the starting concentration was 5 μM (approximately 1629 ng/ml) MDZ.
\hat{ }Data represent the average of six samples of a representative experiment with standard deviation.
^{@}p-values were derived using a T-test.

TABLE 4

| | P-Values^{#} | | | |
|---|---|---|---|---|
| Cell lines | Substrates | | Products | |
| Compared | Tac | MDZ | 1-OH MDZ | 4-OH MDZ |
| ¹HuH-7 *3/*3 vs. | 5.4 × 10⁻³ | 2.4 × 10⁻⁵ | 2.3 × 10⁻⁶ | 7.3 × 10⁻⁴ |
| ²CYP3A5 *1/*3 sd | | | | |
| HuH-7 *3/*3 vs. | 2.7 × 10⁻³ | 5.3 × 10⁻⁴ | 8.6 × 10⁻⁵ | 2.7 × 10⁻⁴ |
| ³CYP3A5 *1/*1 dd | | | | |
| HuH-7 *3/*3 vs. | 2.7 × 10⁻² | 5.0 × 10⁻⁷ | 3.8 × 10⁻⁸ | 1.3 × 10⁻³ |
| ⁴CYP3A5 *1/*3 pm | | | | |
| CYP3A5 *1/*3 sd vs. | 1.0 × 10⁻² | 3.6 × 10⁻¹ | 3.5 × 10⁻² | 1.6 × 10⁻² |
| CYP3A5 *1/*1 dd | | | | |
| CYP3A5 *1/*3 sd vs. | 5.3 × 10⁻² | 5.3 × 10⁻¹ | 1.8 × 10⁻³ | 9.4 × 10⁻¹ |
| CYP3A5 *1/*3 pm | | | | |
| CYP3A5 *1/*1 dd vs. | 3.1 × 10⁻² | 4.1 × 10⁻¹ | 2.0 × 10⁻³ | 2.4 × 10⁻² |
| CYP3A5 *1/*3 pm | | | | |

#p-values were derived using a T-test.
¹HuH-7 was the parental cell line with CYP3A5 *3/*3 alleles
²CYP3A5 *1/*3 sd was a bioengineered cell line with the CYP3A5 *1 allele made by deletion of a splice acceptor on one of the alleles.
³CYP3A5 *1/*1 dd was a bioengineered cell line with both CYP3A5 *1 alleles made by deletion of a splice acceptor on two of the alleles.
⁴CYP3A5 *1/*3 pm was a bioengineered cell line with the CYP3A5 *1 allele made by a point mutation of a splice acceptor on one of the alleles.

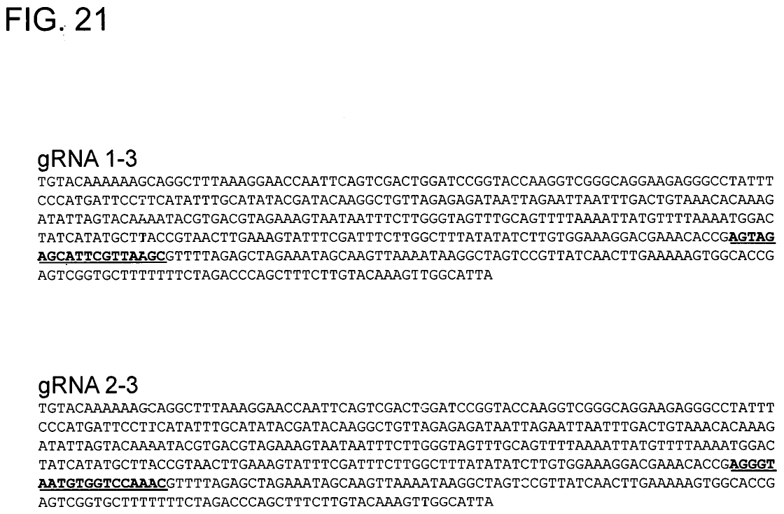

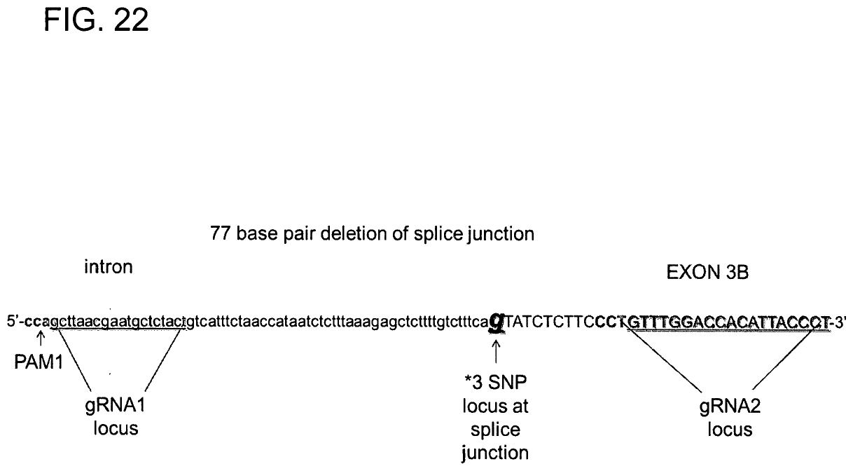

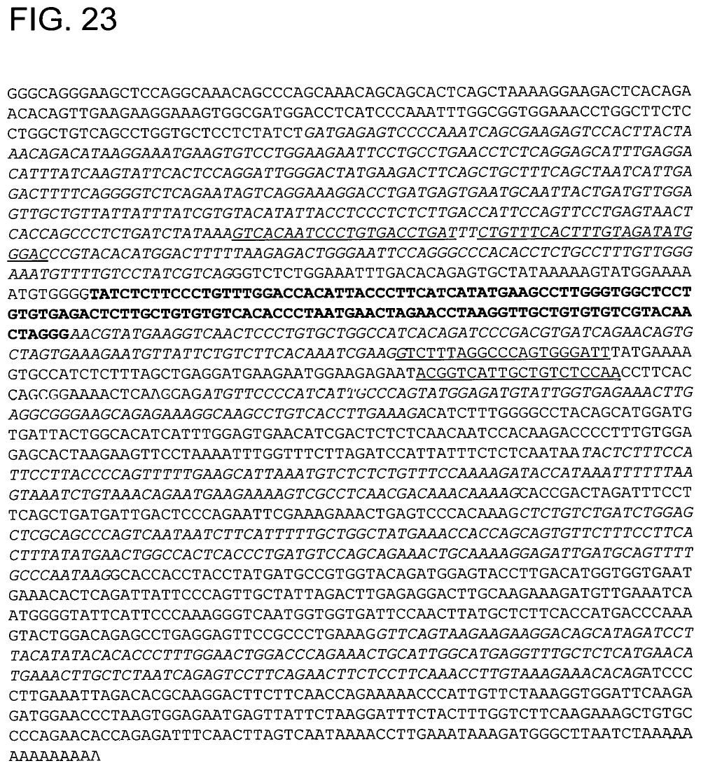

FIG. 21 shows the sequences of the guide RNAs (gRNA) used to modify the CYP3A5 *3 locus in the HuH-7 cell lines. FIG. 22 shows the CYP3A5 *3 locus and where the gRNAs target in the CYP3A5 gene for genetic modification and development of the exemplary cell lines. FIG. 23 shows the CYP3A5 mRNA and the portion of the sequence shown in bold is the Exon 3B that is absent in mRNA expressed from the CYP3A5 *1 expressing allele.

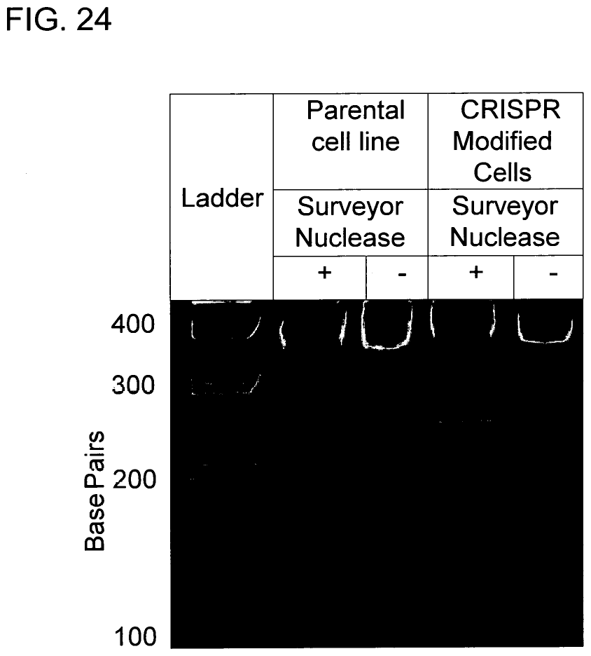

FIG. 24 shows the result of surveyor assay used to assess the guide RNA efficiency for CYP3A5 *3 locus modification. Surveyor nuclease assay indicated HuH-7 parental cells transfected with hCas9, CYP3A5 *3 guide RNA, and HDR template ssODN successfully mutated the CYP3A5 *3 locus. After gRNA treatment, parental and CRISPR-modified genomic DNA was used for PCR and surveyor nuclease detection of genome modification at *3 locus. Parental cell PCR products treated with surveyor nuclease had a 397 bp band, while the CRISPR modified cells' PCR products had bands of 397, 236 and 161 bp indicating genetic modification at the *3 locus in bulk transfected cells.

Table 5 provides other genetic drug metabolism variants CYP3A4*3, CYP3A4*22, CYP3A5 *6 that have been developed for genetic modification.

TABLE 5

| | Chromosomal | Functional | |
|---|---|---|---|
| Variant | location | Consequence | Sequence (5′→3′) |
| rs4986910 | 7:99358524 | Missense | AGTGGACCCA GAAACTGCAT |
| (CYP3A4 *3) | | | TGGCA[C/T]GAGG TTTGCTCTCA |
| | | | TGAACATGAA A (SEQ ID NO: 1) |
| rs35599367 | 7:99366316 | Intron | AGTTCAGTGT CTCCATCACA |
| (CYP3A4 *22) | | variant | CCCAG[C/T]GTAG GGCCAGCTGC |
| | | | ATCACTGGCA C (SEQ ID NO: 2) |
| rs10264272 | 7:99262835 | Synonymous | CTAAGAAACC AAATTTTAGG |
| (CYP3A5 *6) | | codon | AACTT[C/T]TTAG TGCTCTCCAC |
| | | | AAAGGGGTCT T (SEQ ID NO: 3) |

Table 6 shows the sequences of gRNAs created for three other genetic variants. The CYP3A4 *3 gRNAs are ligated to gBLOCKs as prior with the CYP3A5 *3 gRNA and into TOPO vectors. The CYP3A4 *22 and CYP3A5 *6 gRNA oligonucleotides have been ligated into pSpCas9n(BB)-2A-Puro (PX462) and pSpCas9(BB)-2A-GFP (PX458) plasmids for genetic modification. The backbone plasmids are previously described (Ran et al., 2013. Nat Protoc 8(11):2281-2308).

TABLE 6
Variant: rs4986910 (CYP3A4 *3)

| gRNA | Sequence (5′→3′) |
|---|---|
| CYP3A4*3-gRNA 1 | CCAGAAACTG CATTGGCATG AGG |
| | (SEQ ID NO: 47) |
| CYP3A4*3-gRNA 2 | AGTGGACCCA GAAACTGCAT TGG |
| | (SEQ ID NO: 48) |
| CYP3A4*3-g RNA 3 | GTTTGAAGGA GAAGTTCTGA AGG |
| | (SEQ ID NO: 49) |

Variant: rs35599367 (CYP3A4 *22)

| gRNA | Sequence |
|---|---|
| 1-22 ZsgRNA | GATGCAGCTG GCCCTACGCT GGG |
| | (SEQ ID NO: 50) |
| 2-22 ZsgRNA | TCTCCATCAC ACCCAGCGTA GGG |
| | (SEQ ID NO: 51) |

Variant: rs10264272 (CYP3A5 *6)

| gRNA | Sequence |
|---|---|
| 3A5 1-6 ZsgRNA | CAATCCACAA GACCCCTTTG TGG |
| | (SEQ ID NO: 52) |
| 3A5 2-6 ZsgRNA | ACTAAGAAGT TCCTAAAATT TGG |
| | (SEQ ID NO: 53) |
| 3A5 3-6 ZsgRNA | CTTCTTAGTG CTCTCCACAA AGG |
| | (SEQ ID NO: 54) |

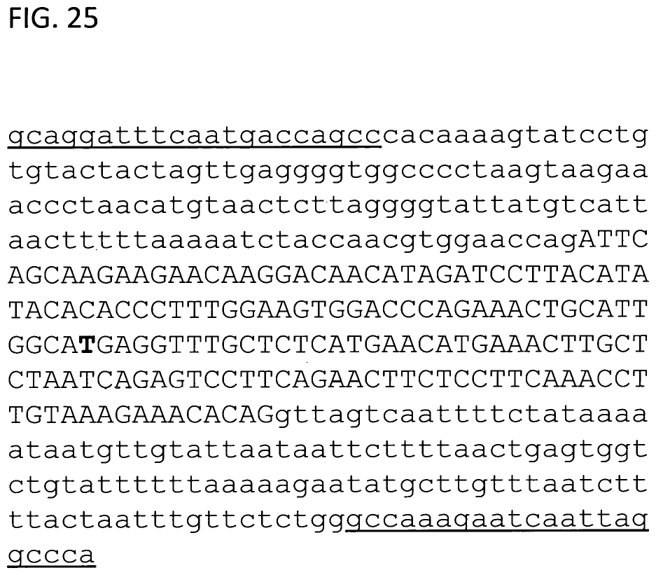

FIG. 25 shows the nucleotide sequence (SEQ ID NO:45) in the CYP3A4 gene surrounding CYP3A4*3 locus (rs4986910) that was targeted for development of genetically modified HuH-7 cells. The bold T in the sequence is the CYP3A4 *3 locus. This locus is associated with tacrolimus metabolism in Caucasians (Jacobson et al., 2015, P, Miller M, Am J Transplant. 15 (suppl 3).

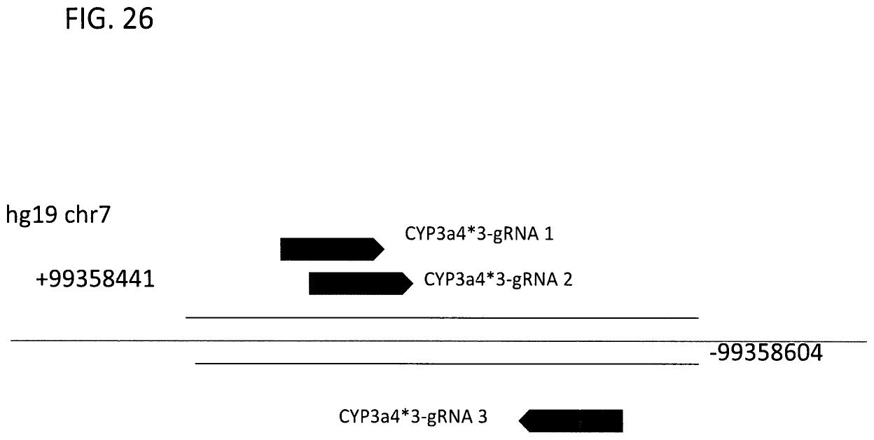

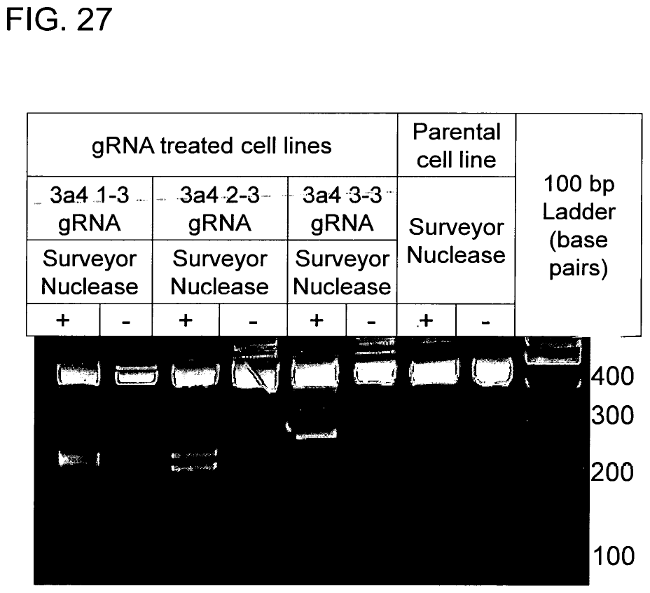

FIG. 26 shows a map of the three gRNAs designed to target the CYP3A4*3 locus. FIG. 27 shows the result of surveyor assay for CYP3A4*3 locus in HuH-7 cell line to assess the effect of genetic modification with three different guide RNAs designed to target the CYP3A4*3 locus. After gRNA treatment, parental and CRISPR-modified genomic DNA was used for PCR and surveyor nuclease detection of genome modification at CYP3A4*3 locus. Nuclease cleaved the genetically modified locus with three guide RNAs treated genomic DNA effectively producing the desired PCR products. The parental cell line had the 437 bp band. Each gRNA-treated cell line has three PCR products, indicating a mixture of genetically-modified cells and parental cells in the bulk transfected cells, thereby demonstrating construction of cell lines that have genetic variants in the gene CYP3A4 at the *3 locus.

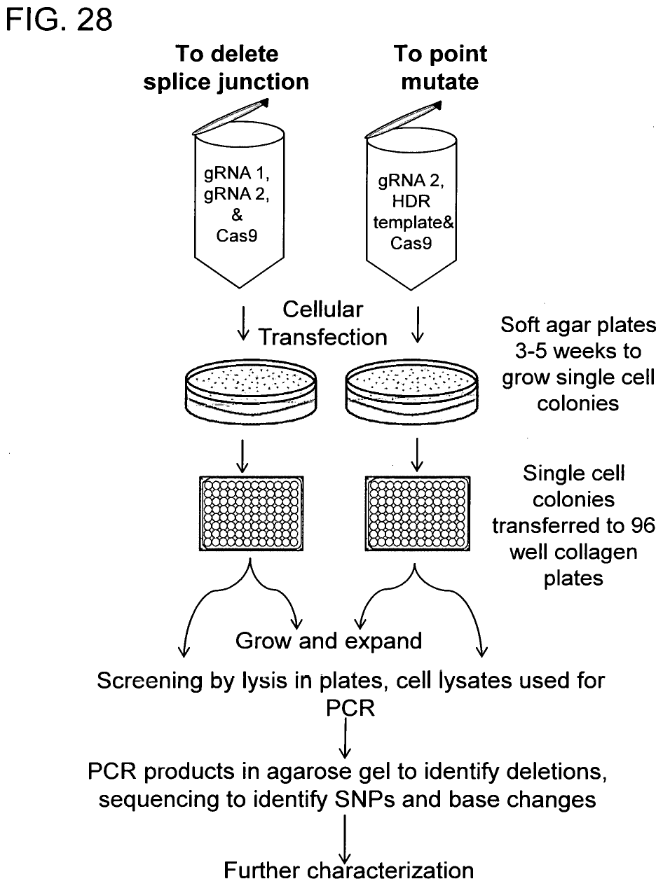

FIG. 28 provides a flow chart of an exemplary process for producing a genetically-modified cell line and then using the variant cell line to evaluate drug metabolism. Generally, the process involves generating genetic variant cell lines that metabolize a drug of interest in a manner similar to the cells of a human genotype. For example, CYP3A5 *1/*1 dd mimics metabolism of drugs such as midazolam or tacrolimus by individuals homozygous for the CYP3A5 *1 allele.

In general, these data allow one to evaluate the effects of genotype on midazolam metabolism. The effects can be considered when determining an appropriate dose of MDZ to be given to a patient having a genotype that corresponds to one of the cell lines—either the parental HuH-7 cell line or one of the genetically-modified cell lines.

Using assays such as this, the effect of a specific genetic variant on drug metabolism can be determined. Genetic variant cell lines can be tested in cell culture to determine a rate of metabolism. This method can speed drug testing methods as it limits the need for excessive clinical trial in humans and can lead to a better starting dose for new drugs and genetic variants. Thus, while described above in the context of specified genetic variants at a specified locus and in the context of metabolizing the specified drug, the methods described can be used for generating additional cell lines with genetic variation at different loci to study metabolism of different drugs.

These data can then be used to determine an appropriate dose of drug to be given to a patient having a genotype that corresponds to one of the cell lines—either the parental cell line or a genetically-modified cell line.

The process described above focuses on genetic variants influencing drug metabolism. Previous work in this area has focused mostly on cancer, using commercially-available panels of cells, typically rat cells or animal models, and employs a reporter gene. In contrast, the methods described herein use genetically-modified cells that correspond to existing human genotypes to study metabolism of drugs without relying on a reporter gene system. That is, the methods described herein modify endogenous genes rather than adding a heterologous gene to the cell. Moreover, the genetic modifications produced by the methods described herein are not limited to knock out mutations. Rather, the genetic modifications can be introduced onto a single chromosome or multiple chromosomes. In some cases, these genetic modifications may cause a complete loss of function of certain genes, but in other cases, the genetic modifications may decrease function to a degree less than complete loss of function or increase function.

The endogenous chromosomes that are subject to genetic modification by the methods described herein are not limited to chromosomes in the nucleus, but can include chromosomes in the mitochondria, genetic material that has been integrated in to the cells, or any other genetic material in the cells. While not limited to the use of human cells, the working examples depart from conventional methods by using human cell lines.

In another aspect, this disclosure describes a process to test genetic variant cell lines in cell culture without the need to conduct extensive clinical tests on humans. Using the genetic variant cell lines can save time and expense in developing dosing guidelines for drugs based on genotype. Furthermore, this process uses cultured, immortalized cell lines, which make genetic variant testing much more sustainable compared to using primary cell or liver specimens. Additionally, testing genetic variants in cell lines reduces environmental influences by containing the cells in a lab. Furthermore, the parental cells can be genetically similar to the variant cell lines—i.e., the specific genetic variant being investigated in the variant cell line can be the only genetic difference between the variant cell line and the parental cell line.

For example, since the CYP3A5 *1/*1 dd cell line has the most midazolam and tacrolimus metabolic activity, one can use CYP3A5 *1/*1 dd cell line as a parental cell line for further genetic variant testing using this process. CYP3A5 *1/*1 dd cell line also has exceptional metabolic activity for metabolizing Tac and MDZ. Thus, this process is unique compared to traditional methods of investigating genetic variants effect on drug metabolism. CYP3A5 *1/*1 dd would represent a common genotype in African Americans and thus would be very useful for studying genetic variants in African Americans.

Common variants, such as CYP3A5 *1, *3, *6, *7 are associated with tacrolimus metabolism and other drugs. However, common variants in CYP3A5, along with clinical factors, only account for ˜50% of the tacrolimus variability in the blood of African American kidney transplant recipients. Thus, rare variants, with low frequency within populations, are likely to account for the remaining variability in tacrolimus blood concentrations in patients. This is similar for other drugs that go through drug metabolism pathways in which genetic variation affects the metabolism and effective blood concentrations of drugs. Thus, the methods described herein can be used to generate genetically-modified useful for evaluating drug metabolism for both common and rare variants. The evaluations allow one to better understand the effect that a genetic variant can have on drug metabolism, which can then be translated to better human dosing for drugs, where the human subject possesses a genotype that metabolizes a particular drug in manner similar to the genetically-modified cell line generated as described herein. For example, the presence of one CYP3A5 *1 allele in kidney transplant patients was associated with a 36% reduction in log-transformed dose-normalized troughs (0.44 0.03 [SE]), while a 59% reduction was observed if two CYP3A5 *1 alleles were present (Jacobson et al., 2011. Transplantation 91: 300-308). The drug metabolism in these patients can be approximated using cell models as described herein.

These genetic variants are likely to be in other drug metabolism and drug metabolism associated genes, such as those listed in Table 7.

TABLE 7

| Gene | Protein name | Function |
|---|---|---|
| CYP3A locus | Cytochrome P450 subfamily: | Drug and substrate metabolism. |
| | CYP3A4, CYP3A5, CYP3A43, | |
| | CYP3A7, CYP3AP1 | |
| CYP2J2 | Cytochrome P450, subfamily 2J | P450 enzyme expressed in intestine, heart. |
| | polypeptide 2 | Drug metabolism. Metabolizes arachidonic |
| | | acid promoting kidney homeostasis. |
| CYP2A6 | Cytochrome P450 2A6 | Drug and substrate metabolism. |
| CYP2B6 | Cytochrome P450 2B6 | Drug and substrate metabolism. |
| CYP2E1 | Cytochrome P450 2E1 | Drug and substrate metabolism. |
| CYP2C8 | Cytochrome P450 2C8 | Drug and substrate metabolism. |
| CYP2C19 | Cytochrome P450 2C19 | Drug and substrate metabolism. |
| CYP1A2 | Cytochrome P450 1A2 | Drug and substrate metabolism. |
| CYP2D6 | Cytochrome P450 2D9 | Drug and substrate metabolism. |
| CYP2C9 | Cytochrome P450 2C9 | Drug and substrate metabolism. |
| Co-enzymes | | |
| POR | cytochrome P450 oxidoreductase | P450 oxidoreductase and reduced cytochrome |
| | | b5 supply electrons into the P450 cycle. |
| | | Addition of cyt b5 stimulates CYP3A4 |
| | | activity in vitro. |
| | | Oxidoreductase responsible for electron |
| | | transfer from NAD to CYP450, (POR*28 is |
| | | associated with increased CYP3A activity and |
| | | increase clearance of tacrolimus. |
| CYB5A | Cytochrome B5, TypeA | Participant in the CYP450 cycle as an |
| | | electron donor for cytochrome b5. Drug . |
| | | metabolism |
| CYB5R1 | NADH-Cytochrome B5 | Reduces cytochrome b5. Cytochrome b5 |
| | Reductase | donates second electron in P450 cycle and |
| | | enhances CYP3A activity. |
| CYB5R2 | NADH-Cytochrome B5 | Bifunctional reductase that contains |
| | Reductase-2 | cytochrome b5 and reductase domains in |
| | | same protein. Cytosolic enzyme. |
| CYB5R3 | Cytochrome B5 Reductase 3 | Participant in CYP450 cycle as electron |
| | | donor for cytochrome b5. Drug |
| | | metabolism. Present in endoplasmic |
| | | reticulum membrane. |
| CYB5R4 | NADH-Cytochrome B5 | Reduces cytochrome b5. Cytochrome b5 |
| | Reductase-4 | supplies second electron in P450 cycle and |
| | | stimulates CYP3A activity. |
| CYB5RL | NADH-Cytochrome B5 | Reduces cytochrome b5. |
| | Reductase-Like | |
| CYB5D1 | Cytochrome B5 Domain- | Serves as an electron donor for cytochrome |
| | Containing Protein-1 | b5 and thus participates in CYP450 cycle. |
| | | Drug metabolism. |
| Transporters | | |
| ABCB1 (MDR1) | ATP-Binding Cassette, Subfamily | Efflux transporter known as Multi Drug |
| | B, member 1 | Resistance1 or P-glycoprotein. Tacrolimus is |
| | | a substrate. Actively transports tacrolimus |
| | | into the intestinal lumen as a counter- |
| | | transport pump. |
| ABCC1 (MRP1 ) | ATP-Binding Cassette, subfamily | Efflux transporter. Also known as Multidrug |
| | C, member 1 | resistance associated protein 1 (MRP1). |
| | | Findings suggest that MDR1 polymorphisms |
| | | has effect on tacrolimus pharmacodynamics. |
| ABCC2 (MRP2) | ATP-Binding Cassette, subfamily | Efflux transporter also known as Multidrug |
| | C, member 2 | resistance associated protein 2 (MRP2). |
| ABCG2 | ATP-Binding Cassette, Subfamily | Efflux transporter, also named Breast Cancer |
| | G, member 2 | Resistance Protein. Tacrolimus is an inhibitor. |
| | | Variants in ABC transporter gene may also |
| | | associate with tacrolimus pharmacokinetics. |
| ABCE1/RNS4I | ATP-Binding Cassette, Subfamily | Efflux transporter also known as ribonuclease |
| | E, member 1 | 4 inhibitor. |
| SLCO1B3 | Solute Carrier Organic anion | Uptake transporter for organic anions. Also |
| | transporter family, member 1B3 | known as OATP1B3. |
| Transcription Factors | | |
| VDR | Vitamin D Receptor | Ligand activated transcription factors) that |
| | | control gene expression). Highly expressed |
| | | in intestine, but not in liver. Affects |
| | | intestinal expression of CYP3A. |
| NR3C1 (GR) | Nuclear Receptor Subfamily 3, | Glucocorticoid Receptor. Glucocorticoid- |
| | group Member 1 | activated transcription factor that controls |
| | | gene expression (several drug metabolizing |
| | | genes contain GR response elements). |
| NR1I2 (PXR) | Nuclear Receptor Subfamily 1, | Pregnane X Receptor. Ligand activated |
| | group 1, Member 2 | transcription factors) that control gene |
| | | expression. Regulates expression of drug |
| | | metabolizing enzymes and drug transporters |
| | | in liver. |
| NR1I3 (CAR) | Nuclear Receptor Subfamily 1, | Constitutive Androstane Receptor. Ligand- |
| | group 1, Member 3 | activated transcription factors) that control |
| | | gene expression. Alters expression of CYP3A |
| | | genes. Key regulator of drug metabolizing |
| | | enzymes and drug transporters. |
| HNF4A | Hepatocyte Nuclear Factor-4-α | Transcription factor for hepatic gene |
| | | expression regulation. Regulates PXR and |
| | | CAR expression and CYP3A expression. |
| CEBPA | C/EBP-Alpha | Co-factor (activator) for gene regulation. |
| | | Especially transporters ABBC2 and ABCB1. |
| CEBPB | CCAAT/Enhancer Binding | Co-factor (activator) for gene regulation. |
| | Protein, Beta | Especially transporters ABBC2 and ABCB1. |
| PPARA | Peroxisome Proliferator-Activator | Has regulatory effect on CYP3A4 expression. |
| | Receptor Alpha | |
| FOXA2 | Forkhead Box protein A2 | Transcription factor also named HNF3-β, has |
| | | effect on hepatic CYP3A4 expression. |
| NCOR1 | Nuclear Receptor Corepressor 1 | Co-factor (repressor) for gene regulation. |
| | | Associated with transporters ABBC2 and |
| | | ABCB1. |
| YY1 | Transcriptional Repressor Protein | Downregulates Cytochrome c Oxidase and |
| | | CYP3A4 and CYP3A5. |

Thus, in one aspect, this disclosure describes a method for making a cell line that approximates metabolism of a drug by a human patient—i.e., metabolizes a drug in a manner similar to the human patient—in which genotype of the human patient affects metabolism of the drug. Generally, the method includes identifying a locus and allelic variants in cells of the human patient that affect metabolism of the drug, providing a parental cell line that possesses the locus that affects metabolism of the drug in the cells of the patient, and introducing a genetic modification into the parental cell at the locus, thereby forming a variant cell line comprising an allelic variant at the locus having the genetic modification.

In some cases, one can analyze metabolism of the drug by the variant cell line compared to a human homozygous for the wild-type allele at the locus. In some cases, one can analyze metabolism of the drug by the variant cell line compared to a human that possesses at least one non-wild-type allelic variant. In either case, the identity of the wild-type allele or a non-wild-type allele will be determined by the particular locus of interest. The locus of interest may be determined, at least in part, by the drug whose metabolism is of interest.

A cell line that approximates metabolism of the drug by a patient having a known genotype at the locus may be identified as a model cell line that can represent the corresponding genotype when analyzing metabolism of the drug in vitro. In vitro analysis of drug metabolism using a model cell line allows one to assess whether a standard dose of the drug is appropriate for a patient having the genotype that corresponds to the model cell line.

As used herein, a model cell line “approximates metabolism” of a drug by a reference—e.g., a human patient or cells from a human patient—if the cell line metabolizes (or clears) drug from culture medium in vitro at a rate that approximates that rate at which a patient having a known genotype metabolizes (or clears) the drug from circulation. Clearance of drug from the circulation is more complex and dependent upon many more variables than metabolism of the drug in cell culture. Nevertheless, cell lines generated as described herein can be used as an in vitro model for determining the dose of a drug appropriate for a patient having a genotype that affects metabolism. The cell lines may be useful for providing a qualitative adjustment of a baseline dose—i.e., increase or decrease the baseline dose based on metabolism of the model cell lines that approximates metabolism of the drug by the patient. In some cases, the cell lines may be further useful for providing guidance regarding the degree to which the dose should be adjusted.

As used herein, the term “wild type” refers to the most common allelic variant at a locus in the general human population. The “wild type” genotype may therefore differ from the most common genotype of a particular subpopulation.

It is possible to develop multiple cell lines in which the genetic modification at the locus that affects metabolism of the drug in one cell line is different than the genetic modification at the locus in a second cell line. Where different cell lines possess different genetic modifications, the cell lines may exhibit the same, similar, or different metabolism of the drug. For example, it may be possible to generate two different cell lines, each possessing a different genetic modification at the locus that affects metabolism of the drug, where the different genetic modifications nevertheless produce the same change in metabolism of the drug.

It is also possible to generate a cell line that possesses genetic modifications at more than one locus in which the genotype affects metabolism of the drug. Cell lines that possess genetic modifications at ore than one locus that affects metabolism of a drug can be used to examine the combinatorial interaction of the loci on the metabolism of a drug of interest.

Thus, this disclosure further describes a method of providing personalized dosing of a drug to a patient whose genotype at a locus affects metabolism of the drug. Generally, the method includes determining the genotype of the patient at the locus, culturing a model cell line that approximates metabolism of the drug by a person having the patient's genotype at the locus, evaluating metabolism of the drug by the cell line, and administering to the patient an appropriate dose of the drug based on metabolism of the drug by the cell line.

In some cases, the genotype of the patient can be common—i.e., present in at least 5% of the general human population. In other cases, the patient's genotype can be rare—i.e., present in less than 5% of the general human population. Thus, again, a genotype that may be present in greater than 5% of a particular subpopulation may nevertheless be classified at “rare” if present in less than 5% of the general human population.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Table 8 provides primers used to generate and characterize genetic modifications in the CYP3A4 and CYP3A5 genes. These sequences targeted the gene CYP3A4 at the *3 (rs4986910) or *22 (rs35599367) loci. The primers also target CYP3A5 at the *3 (rs776746), *6 (rs10264272) and *7 (rs41303343) loci.

TABLE 8
Primer used

| | | | SEQ |
|---|---|---|---|
| Gene/ | | | ID |
| Locus | Primer | Sequence (5′→3′) | NO: |
| CYP3A4 | *22F | AATTCTGCTG TCAGGGCAAC | 4 |
| *22 | *22R | TTGAGAGAAA GAATGGATCC AAAAA | 5 |
| | *224F | GGCATAGAGT CTGCAGTCAG G | 6 |
| | *22-1R | TCACCTTCTA TCACACTCCA TCA | 7 |
| | *22-2F | TCAGTGTCTC CATCACACCC | 8 |
| | *22-2R | GGATTGTTGA GAGAGTCGAT GTT | 9 |
| CYP3A4 | 3A4-23, 871F | TTCCATCAGT CAGTCCCATC AC | 10 |
| *3 | 3A4-24, 740R | CAAGTTCTGG TTGGGAAGAG C | 11 |
| | 3A4-24067F | GCAGGATTTC AATGACCAGC C | 12 |
| | 3A4-24502R | TGGGCCTAAT TGATTCTTTG GC | 13 |
| CYP3A5 | 8F | CTGTCAGAGG GGCTAGAGGT | 14 |
| *3 | 8R | CCTCCCAGGT TCAAGCGATT | 15 |
| | 7853F | GCATTTAGTC CTTGTGAGCA CTTG | 16 |
| | 8303R | CATACGTTCT GTGTGGGGAC AAC | 17 |
| | 7884F | ACCTGCCTTC AATTTTTCAC TG | 18 |
| | 8267R | CTTCACTAGC CCGATTCTGC | 19 |
| | 3A5 ex2F | GTCACAATCC CTGTGACCTG AT | 20 |
| | 3A5 ex5R | TTGGAGACAG CAATGACCGT | 21 |
| | 3A5 ex2F Seq | CTGTTTCACT TTGTAGATAT GGGAC | 22 |
| | 3A5 ex5R Seq | AATCCCACTG GGCCTAAAGA C | 23 |
| | Cel1F | CAACTGCCCT TGCAGCATTT | 24 |
| | Cel1R | ACCCAGGAAG CCAGACTTTG | 25 |
| CYP3A5 | 4F | TCTGCCCATC TGTCACCAAT | 26 |
| *6 | 4R | TTGGCCACAT GTCCAGTACT | 27 |
| | 15488F | GGCACCAGAT AACCACCTTC | 28 |
| | 15989R | GGGCTCTAGA TTGACAAAAA CA | 29 |
| CYP3A5 | 12F | TCCTCCACAC ATCTCAGTAG GT | 30 |
| *7 | 12R | TAAGGCCTGA CCTTGTCCCT | 31 |
| | 28064F | ACTTCACGAA TACTATGATC | 32 |
| | | ATTTACC | |
| | 28351R | CATTGACCCT TTGGGAATGA | 33 |

### Parental Cell Line

HuH-7 cells, hepatoma cells from a 57-year-old Japanese male (Japanese Cell Research Bank; cat. # JCRB0403) were used as the parental cell line for genetic modification. Cells were grown in Dulbeco's Modified Eagle Media (DMEM) with high glucose and pyruvate (Gibco, Thermo Fisher Scientific, Inc., Waltham, Mass.) supplemented with 10% Fetal Bovine Serum (Gibco, Thermo Fisher Scientific, Inc., Waltham, Mass.) and Antibiotic-Antimycotic (Gibco, Thermo Fisher Scientific, Inc., Waltham, Mass.). We refer to this media as DMEM throughout.

### Process to Make Guide RNAs Targeting CYP3A5 *3 Locus

Guide RNAs (gRNAs) targeting the CYP3A5*3 locus (rs776746) were designed using the CRISPR design tool (crispr.mit.edu/). The gBLOCK sequences for guide RNA 1-3 (gRNA 1-3) and guide RNA 2-3 (gRNA2-3) shown in FIG. 21 were ordered from Integrated DNA Technologies, Inc. (Coralville, Iowa) and were inserted into the pCR-Blunt II-TOPO vector (Invitrogen, Thermo Fisher Scientific, Carlsbad, Calif.) and then propagated in ONE SHOT TOP10 chemically competent E. coli cells (Invitrogen, Thermo Fisher Scientific, Carlsbad, Calif.) as previously described (Mali et al., 2013. Science 339(6121)823-826). A plasmid that expressed a human codon-optimized Cas nuclease (hCas9) was purchased from Addgene (cat. #41815; Cambridge, Mass.) and expanded in Stbl3 E. coli cells (Invitrogen, Thermo Fisher Scientific, Carlsbad, Calif.). The gRNAs target the CYP3A5 *3 locus as shown in reference to FIG. 22.

Plasmids were then prepared for transfection using the PLASMID MAXI KIT (Qiagen, Hilden, Germany). pCR-Blunt II-TOPO derivative vector plasmids were sequence verified at the University of Minnesota Genomics Center using M13 primers. Plasmids were quantified and assessed for purity using a NanoDrop 2000 UV/Vis spectrophotometer. Plasmids were transfected into the HuH-7 cells using a NEON transfection system (Invitrogen, Thermo Fisher Scientific, Carlsbad, Calif.) following the manufacturer's protocol.

Transfections of DNA into Cells for Genetic Modification of CYP3A5 *3 Locus

To create the CYP3A5 *1/*3 sd and CYP3A5 *1/*1 dd cell lines, HuH-7 cells were transfected with 1-3 gRNA, 2-3 gRNA and hCas9 plasmids. To create CYP3A5 *1/*3 pm, HuH-7 cells were transfected with 2-3 gRNA, and hCas9 plasmids along with HDR single stranded DNA template ssODN 3A5*3_E+. The sequence of ssODN 3A5*3_E+ is 5′-gcttaacgaatgctctactgtcatttctaaccataatctctttaaagagctcttttgtctttcaaTATCTCTTCCGTGTTTGGACCACATTACCCTTCATCATATGAAGCCTTGGGTGGCTCC-3′ (SEQ ID NO:46). The underlined a in the sequence indicates the *3 base that is changed in CYP3A5 *1/*3 pm from a guanine (g).

Cells of the three exemplary cell lines were then plated in DMEM and allowed to grow for 1-2 weeks with periodic changing of the DMEM until the cells were nearing confluence in a 10-cm² tissue culture dish. CYP3A5 *1/*3 pm was cultured in the presence of L755,507 (Xcessbio Biosciences, Inc., San Diego, Calif., catalogue Number M60237-2s) and SCR7 (Xcessbio Biosciences, Inc., San Diego, Calif., catalogue Number M60082-2) to promote Homology Directed Repair.

### Surveyor Assay to Select Guide RNAs

Genomic DNA was isolated from transfected cells and then a Surveyor assay was performed to screen gRNAs for ability to cut at CYP3A5 *3 locus as previously described (Guschin et al., 2010. Methods Mol Biol 649:247-256) along with Surveyor enzyme (Integrated DNA Technologies, Inc., Coralville, Iowa). Briefly, DNA was extracted from bulk transfected cells using High-Pure PCR template preparation kit (Roche Diagnostics, Corp., Indianapolis, Ind.). PCR was performed using ACCUPRIME Taq DNA Polymerase (Thermo fisher Scientific, Inc., Waltham, Mass.), high fidelity with CYP3A5 specific primers Cel1F*3 (5′-CAACTGCCCTTGCAGCATTT-3′; SEQ ID NO:24) and Cel1R*3 (5′-ACCCAGGAAGCCAGACTTTG-3′; SEQ ID NO:25) to produce a 397 bp product (if no deletions). The PCR thermocycler (Bio-Rad Laboratories, Inc., Hercules, Calif.) was programmed as:

1. 94° C. for 5 minutes,

2. 94° C. for 15 seconds,

3. 56° C. for 30 seconds,

4. 68° C. for 30 seconds,

5. Repeat 2-4 34 times,

6. 68° C. for 30 seconds,

7. 4° C. indefinitely.

PCR products were denatured and re-annealed as previously described (Guschin et al., 2010. Methods Mol Biol 649:247-256) and visualized in a 10% CRITERION TBE polyacrylamide gel (Bio-Rad Laboratories, Inc., Hercules, Calif.) to determine DNA heteroduplexes from heterogeneous cell cultures caused by CRISPR/Cas9 and gRNA targeting CYP3A5 *3 locus.

### Single-Cell Cloning to Isolate Homogenous Cell Lines

Transfected cells were also plated in media/soft agar mixture as previously described (Dorr et al., 2015. Mol Cancer Res 13(8):1238-1247) and propagated to become homogenous cell lines. Specifically, 15,000 cells were plated between layers of a 0.6% and a 0.3% solution of ULTRAPURE LMP Agarose (Invitrogen, Thermo Fisher Scientific, Carlsbad, Calif.) diluted in DMEM and incubated at 37° C. with 5% CO₂ for about five weeks. Colonies were transferred to individual wells of a 96-well round bottom plate and cultured until confluent.

### Screening of Cell Lines

For large scale screening, we dissociated the cells using Trypsin-EDTA (0.25%), phenol red (Gibco, Thermo Fisher Scientific, Inc., Waltham, Mass.) and transferred half the culture to fresh 96-well plates with DMEM. These cells were placed back in the incubator to grow at 37° C. with 5% CO2. The remaining cells in the 96-well plate were centrifuged at 350×g for five minutes. Trypsin was removed and the cell pellets were lysed using the QUICKEXTRACT DNA extraction solution (Lucigen Corp., Middleton, Wis.). Lysates were used as PCR template and then PCR amplified with an ACCUPRIME Hi Fidelity PCR kit (Invitrogen, Thermo Fisher Scientific, Carlsbad, Calif.) in 96-well PCR plates. The primers for amplification of the CYP3A5*3 region were designed using NCBI Primer design (ncbi.nlm.nih.gov/tools/primer-blast/) and are:

7853F
(5′-GCATTTAGTCCTTGTGAGCACTTG-3′; SEQ ID NO: 16)
and
8303R
(5′-CATACGTTCTGTGTGGGGACAAC-3′; SEQ ID NO: 17).

The thermocycler was programmed as follows:

1. 94° C. for 5 minutes,

2. 94° C. for 15 seconds,

3. 55° C. for 30 seconds,

4. 68° C. for 30 seconds,

5. Repeat steps 2-4, 34 times,

6. 68° C. for 7 minutes,

7. 4° C. indefinitely.

PCR products were purified using the MINELUTE 96 UF PCR purification kit (Qiagen, Hilden, Germany), characterized by electrophoresis through a 2% agarose gel and by sequencing of the PCR products.

Sequencing was performed by the University of Minnesota Genomics Center using sequencing primer 7884F (5′-ACCTGCCTTCAATTTTTCACTG-3′; SEQ ID NO:18). Sequences were analyzed using LASERGENE (DNASTAR, Inc., Madison, Wis.) software and/or GENEIOUS software (Biomatters Ltd., Auckland, NZ) to detect mutants. Cell lines of interest (including, but not limited to, CYP3A5 *1/*3 sd (PTA-123710), CYP3A5 *1/*1 dd (PTA-124693), and CYP3A5 *1/*3 pm (PTA-124694)) were propagated for further analysis and characterization. Sequences of selected example cell lines are illustrated in FIG. 4 and the PCR products are in FIG. 5.

### RNA Splicing Assay

RNA was isolated from the confluent cells using the RNeasy Mini Kit (Qiagen, Hilden, Germany). RNA was quantified using the QUBIT 2.0 fluorimeter (Invitrogen, Thermo Fisher Scientific, Inc., Carlsbad, Calif.) and the QUBIT RNA BR assay kit (Invitrogen, Thermo Fisher Scientific, Inc., Carlsbad, Calif.). RNA was then converted to cDNA using oligo-dT primer and THERMOSCRIPT reverse transcriptase kit (Invitrogen, Invitrogen, Thermo Fisher Scientific, Inc., Carlsbad, Calif.) following the manufacturer's directions. CYP3A5 *1/*3 or CYP3A5 *1/*1 genotyped human liver RNA was also used as controls and cDNA made by same method (samples provided by Erin Schuetz, St. Jude Medical Center).

PCR primers were developed using the NCBI primer design tool and mRNA sequence shown in FIG. 23 of CYP3A5 of the 500 base pairs surrounding the *3 nucleotide locus (Genbank accession # BC025176.1). The cDNA was then used as PCR template with primers: CYP3A5 cDNA ex2F (5′-GTCACAATCCCTGTGACCTGAT-3′; SEQ ID NO:20) and CYP3A5 cDNA ex5R (5′-TTGGAGACAGCAATGACCGT-3′; SEQ ID NO:21) using the ACCUPRIME Hi Fidelity PCR kit (Invitrogen, Thermo Fisher Scientific, Carlsbad, Calif.).

Thermocycler settings were as follows:

1. 94° C. for 5 minutes,

2. 94° C. for 15 seconds,

3. 50.5° C. for 30 seconds,

4. 68° C. for 30 seconds,

5. Repeat steps 2-4 34 times

6. 68° C. for 5 minutes,

7. 4° C. indefinitely

RT-PCR products were then purified using the QIAQUICK PCR purification kit (Qiagen, Hilden, Germany) then characterized by electrophoresis through a 2% agarose gel and imaged using ethidium bromide staining and a CHEMIDOC touch imaging system (Bio-Rad Laboratories, Inc., Hercules, Calif.). These RT-PCR products were then sequenced at the University of Minnesota Genomics Center using the primers CYP3A5 cDNA ex2Fseq (5′-CTGTTTCACTTTGTAGATATGGGAC-3′; SEQ ID NO:22) and CYP3A5 cDNA ex5Rseq (5′-AATCCCACTGGGCCTAAAGAC-3′; SEQ ID NO:23)

### Midazolam and Tacrolimus Metabolism Assays

HuH-7 and engineered cells were grown to confluence in 12 well BIOCOAT collagen I multi-well plates (Corning, Inc., Corning, N.Y.) for 3-4 weeks in DMEM. Cells were then overlaid with MATRIGEL matrix (Corning, Inc., Corning, N.Y.) and then induced for four days by adding 100 μM phenytoin (USP) and 10 μM rifampicin to the cell culture, with media changed daily with inducers. On the fourth day, 500 μl of media was added to the cells with 100 μM phenytoin (USP) and 10 μM rifampicin with either equal volumes of methanol, as a negative control, or Midazolam (MDZ; Cerrilliant Corp., Round Rock, Tex.) diluted in methanol so that MDZ final concentration was 5 μM in cell culture media. Cells were incubated overnight, and media was collected and assayed for MDZ, 1-OH MDZ and 4-OH MDZ by high performance liquid chromatography.

To determine the metabolic function of the engineered cell lines, the cells were treated with MDZ at 1628 ng/mL (5 μM) in cell culture overnight. The following day, the cell culture media was harvested and analyzed by liquid chromatography/mass spectrometry (LC/MS) at the University of Minnesota Department of Experimental and Clinical Pharmacology to quantify the concentrations of MDZ and the metabolites 1-hydroxy-midazolam (1-OH MDZ) and 4-hydroxy-midazolam (4-OH MDZ).

Likewise, Tac was added to cells and analyzed by LC/MS at the University of Minnesota Department of Experimental and Clinical Pharmacology.

### CYP3CIDE Experiments

All CYP3CIDE experiments were done following the aforementioned midazolam metabolism assay with the addition of CYP3CIDE (Sigma-Aldrich, St. Louis, Mo.) resuspended in dimethyl-sulfoxide. 50 μM CYP3CIDE was added to the cells at the same time as the addition of MDZ.

### Metabolism and Tacrolimus Assay Data Analysis

All MDZ and Tac data was graphed using Graphpad Prism statistical software. P-values were determined using a two-tailed, paired, T-Test with Microsoft Excel. Averages and standard deviations were determined using Microsoft Excel.

In application, the present invention is usable to better inform early clinical trials by testing and validating the impact of genetic variants on drug metabolism in cell culture, instead of humans. While the present invention has been described in the context of a genetically modified human liver cell line, it should be understood that the foregoing relates to exemplary embodiments of the invention and that modifications may be made without departing from the spirit and scope of the invention as set forth in the claims above.

### Example 2

### Genotyping of Cell Lines

Genomic DNA was isolated from HuH-7 cells using the HIGH PURE PCR template preparation kit (Roche Diagnostics, Corp., Indianapolis, Ind.). PCR and sequencing primers (Table 9), surrounding SNPs CYP3A4 *22 (rs35599367, C>T), CYP3A5 *3 (rs776746, 6986A>G), *6 (rs10264272, 14690G>A) and *7 (rs41303343, nonfunctional) were designed using the NCBI primer-BLAST primer design tool.

TABLE 9

| | | | SEQ |
|---|---|---|---|
| Gene/ | | | ID |
| Locus | Primer | Primer Sequence 5′→3′ | NO: |
| CYP3A4 | *22F | AATTCTGCTG TCAGGGCAAC | 4 |
| *22 | *22R | TTGAGAGAAA GAATGGATCC AAAAA | 5 |
| | *22-1F | GGCATAGAGT CTGCAGTCAG G | 6 |
| | *224R | TCACCTTCTA TCACACTCCA TCA | 7 |
| | *22-2F | TCAGTGTCTC CATCACACCC | 8 |
| | *22-2R | GGATTGTTGA GAGAGTCGAT GTT | 9 |
| CYP3A5 | 8F | CTGTCAGAGG GGCTAGAGGT | 14 |
| *3 | 8R | CCTCCCAGGT TCAAGCGATT | 15 |
| | 7853 F | GCATTTAGTC CTTGTGAGCA CTTG | 16 |
| | 8303R | CATACGTTCT GTGTGGGGAC AAC | 17 |
| | 7884F | ACCTGCCTTC AATTTTTCAC TG | 18 |
| | 8267R | CTTCACTAGC CCGATTCTGC | 19 |
| | 1A5 ex2F | GTCACAATCC CTGTGACCTG AT | 20 |
| | 3A5 ex5R | TTGGAGACAG CAATGACCGT | 21 |
| | 3A5 ex2F Seq | CTGTTTCACT TTGTAGATAT GGGAC | 72 |
| | 3A5 ex5R Seq | AATCCCACTG GGCCTAAAGA C | 23 |
| | 4F | TCTGCCCATC TGTCACCAAT | 26 |
| | 4R | TTGGCCACAT GTCCAGTACT | 27 |
| | 15488F | GGCACCAGAT AACCACCTTC | 28 |
| | 15989R | GGGCTCTAGA TTGACAAAAA CA | 29 |
| CYP3A5 | 12F | TCCTCCACAC ATCTCAGTAG GT | 30 |
| *7 | 12R | TAAGGCCTGA CCTTGTCCCT | 31 |
| | 28064F | ACTTCACGAA TACTATGATC | 32 |
| | | ATTTACC | |
| | 28351R | CATTGACCCT TTGGGAATGA | 33 |
| GAPDH | GAPDHF | GCATCCTGCA CCACCA | 35 |
| | GAPDHR | GGATGACCTT GCCCACA | 36 |

The sequences surrounding the SNPs in the genomic DNA from the HuH-7 cells were PCR amplified using ACCUPRIME Pfx DNA polymerase kit (Thermo Fisher Scientific, Inc., Waltham, Mass.) and then the PCR products were characterized on 1% agarose gel or purified with a PCR clean up kit (Qiagen, Hilden, Germany) and sequenced. PCR products were then Sanger sequenced using the primers listed in Table 9.

### Plasmids, Guide RNA Construction and Transfection

A plasmid that expressed a human codon-optimized Cas9 (Mali et al., 2013. Nat Biotechnol 31:833-838; Mali et al, 2013. Nat Methods 10:957-963; Mali et al., 2013. Science n339:823-826) nuclease was purchased from Addgene (cambridge, MA). Guide RNAs (gRNAs) targeting the CYP3A5*3 locus were designed using the CRISPR design tool at http://crispr.mit.edu/. DNA gBLOCKS were designed, synthesized, and purchased from Integrated DNA Technologies, Inc. (Coralville, Iowa) combining the gRNA from the CRISPR design tool with the gRNA synthesis protocol previously described (Mali et al., 2013. Nat Biotechnol 31:833-838). The gBLOCKs were TOPO cloned using the Zero Blunt TOPO PCR cloning kit (Invitrogen, Thermo Fisher Scientific, Carlsbad, Calif.) into pCR Blunt II-TOPO vector (Invitrogen, Thermo Fisher Scientific, Carlsbad, Calif.). Plasmids were expanded in ONE SHOT Stbl3 chemically competent E. coli cells (Invitrogen, Thermo Fisher Scientific, Carlsbad, Calif.). Plasmids were sequence verified. Plasmids were then prepared for transfection following a Qiagen Plasmid Maxi Kit (Qiagen, Hilden, Germany). Plasmids were quantified and assessed for purity using a NanoDrop 2000 UV/Vis spectrophotometer. Newly designed gRNAs and hCas9 plasmid DNA were transfected into the HuH-7 cells using a NEON transfection system (Invitrogen, Thermo Fisher Scientific, Carlsbad, Calif.)

### Surveyor Assay to Select Guide RNAs

Surveyor assay was performed as described in Example 1.

Transfection with Selected gRNA and hCAS9

To create cell lines that delete the CYP3A5*3 splice junction, via non-homologous end joining (NHEJ), two selected gRNAs (gRNA1 and gRNA2) and hCas9 plasmids were transfected into the HuH-7 cells using a NEON transfection system (Invitrogen, Thermo Fisher Scientific, Carlsbad, Calif.). The two gRNAs target each side of the CYP3A5*3 locus. The resultant cells were then single cell cloned to produce homogenous cell lines.

To create the single-nucleotide polymorphism (SNP) cell line, gRNA2 was transfected into cells, with homology directed repair (HDR) template. HDR single-stranded DNA template ssODN 3A5*3_E+. The sequence of ssODN 3A5*3_E+ is 5′-gcttaacgaatgctctactgtcatttctaaccataatctctttaaagagctatttgtattcaaTATCTCTTCCGTGTTTGGACCACATTACCCTTCATCATATGAAGCCTTGGGTGGCTCC-3′ (SEQ ID NO:46). The underlined a indicates the *3 base that is changed in from a guanine (g). The lower case letters are the intron sequence, while the upper case letters are the exon 3B sequence. The cells were treated with 1 μM SCR7 (Xcessbio Biosciences, Inc., San Diego, Calif.) and 5 μM L755,507 (Xcessbio Biosciences, Inc., San Diego, Calif.) at the time of transfection and during the seven days following transfection until single-cell cloning.

### Single Cell Cloning and Cell Line Screening Via PCR and DNA Sanger Sequencing

Transfected cells were plated in media/soft agar mixture as previously described (Dorr et al., 2015. Mol Cancer Res 13(8):1238-1247) and propagated to become homogenous cell lines. Specifically, in 150-mm³ 15 ml of a 0.6% solution of ULTRAPURE low melting point agarose (Thermo Fisher Scientific, Inc., Waltham, Mass.) in media was plated at 38.5° C. and cooled until solid. Next, 15,000 transfected cells in 15 mL of 38.5° C. media with 0.3% ULTRAPURE low melting point agarose (Thermo Fisher Scientific, Inc., Waltham, Mass.) was layered on top and cooled. The plate was covered with 10 mL media and incubated at 37° C. with 5% CO₂ for about 3-5 weeks until cell colonies were visible. Colonies were then picked with a sterile 200 μL pipette tip and transferred to individual wells of a 96-well collagen I coated plate and cultured until confluent (approximately 3-4 weeks).

For large scale screening, cells were dissociated using Trypsin-EDTA (0.25%) and half the culture was transferred to fresh 96-well plates with media and grown. The remaining cells in the 96-well plate were centrifuged at 350×g for five minutes. Trypsin was removed and the cell pellets were lysed using the QUICKEXTRACT DNA extraction solution (Lucigen Corp., Middleton, Wis.). Lysates were used as PCR template and then PCR amplified with an ACCUPRIME Pfx DNA polymerase kit (Thermo Fisher Scientific, Inc., Waltham, Mass.) in 96-well PCR plates. The primers for amplification of the CYP3A5 *3 region were designed using NCBI Primer design (ncbi.nlm.nih.gov/tools/primer-blast/) and are: 7853F (5′-GCATTTAGTCCTTGTGAGCACTTG-3′; SEQ ID NO:16) and 8303R (5′-CATACGTTCTGTGTGGGGACAAC-3′; SEQ ID NO:17). Thermocycler was programmed as follows:

1. 94° C. for 5 minutes,

2. 94° C. for 15 seconds,

3. 55° C. for 30 seconds,

4. 68° C. for 30 seconds,

5. Repeat steps 2-4 34 times,

6. 68° C. for 7 minutes,

7. 4° C. indefinitely.

PCR products were purified using the MINELUTE 96 UF PCR purification kit (Qiagen, Hilden, Germany), characterized by electrophoresis through a 2% agarose gel and by sequencing of the PCR products. Sequencing was performed by the UMGC using sequencing primer 7884F (5′-ACCTGCCTTCAATTTTTCACTG-3′; SEQ ID NO:18) and 8267R (5′-CTTCACTAGCCCGATTCTGC-3′; SEQ ID NO:19). Sequence data were analyzed using DNA LASERGENE (DNASTAR, Inc., Madison, Wis.) software and/or GENEIOUS software (Biomatters Ltd., Auckland, NZ).

### RNA Splicing Assay

RNA was isolated from the confluent cells using the RNeasy Mini Kit (Qiagen, Hilden, Germany). RNA was quantified using the QUBIT 2.0 fluorimeter (Invitrogen, Thermo Fisher Scientific, Inc., Carlsbad, Calif.) and the QUBIT RNA BR assay kit (Invitrogen, Thermo Fisher Scientific, Inc., Carlsbad, Calif.). RNA was then converted to cDNA using oligo-dT primer and THERMOSCRIPT reverse transcriptase kit (Invitrogen, Invitrogen, Thermo Fisher Scientific, Inc., Carlsbad, Calif.). CYP3A5 *1/*3 or *1/*1 genotyped human liver RNA was also used as controls and cDNA made by same method. PCR primers were developed using the NCBI primer design tool and mRNA sequence file of CYP3A5 (Genbank accession # BC025176.1) of the 500 base pairs surrounding the *3 nucleotide locus. The cDNA was then used as PCR template with primers: CYP3A5 cDNA ex2F (5′-GTCACAATCCCTGTGACCTGAT-3′; SEQ ID NO:20) and CYP3A5 cDNA ex5R (5′-TTGGAGACAGCAATGACCGT-3′; SEQ ID NO:21) using the ACCUPRIME Pfx DNA polymerase kit (Invitrogen, Invitrogen, Thermo Fisher Scientific, Inc., Carlsbad, Calif.). Thermocycler settings were as follows:

1. 94° C. for 5 minutes,

2. 94° C. for 15 seconds,

3. 50.5° C. for 30 seconds,

4. 68° C. for 30 seconds,

5. Repeat steps 2-4 34 times,

6. 68° C. for 5 minutes,

7. 4° C. indefinitely.

PCR products were purified using the QIAquick PCR purification kit (Qiagen, Hilden, Germany) then characterized by electrophoresis through a 2% agarose gel and imaged using ethidium bromide staining and a CHEMIDOC touch imaging system (Bio-Rad Laboratories, Inc., Hercules, Calif.).

### Quantitative RT-PCR to Detect CYP3A5 Transcripts

CYP3A5 mRNA was quantified using cells from the MDZ and Tac assays. The primers were the same as for the mRNA splicing assay (Table 9). The RNA was the same as the RNA splicing assays and GAPDH primers were used as the reference control. 2 μg of RNA was converted to cDNA and 5 μl of 1-in-20 diluted cDNA was used in a 20 μl reaction mix for SYBR Green assay based quantitative RT-PCR using a LIGHTCYCLER (Roche Diagnostics, Inc., Indianapolis, Ind.). Data were graphed using PRISM software (Graphpad Software, La Jolla, Calif.).

### Immunoblot Analysis for CYP3A5 *1 and CYP3A5 *3 Variants in Engineered Cell Lines

CYP3A4 and CYP3A5 protein expression was determined by immunoblot analysis. Total lysates were recovered from HuH-7 cells and the new derivative cell lines. For microsome preparation, cells were centrifuged at 1500 rpm for five minutes and washed immediately in 1×PBS. This was followed by homogenization using a glass-Teflon homogenizer and a microsome storage buffer (MSB) containing 100 mM potassium phosphate, pH 7.4, 1.0 mM EDTA 20% glycerol with protease inhibitor cocktail. Following differential centrifugation (12,000×g for 30 minutes; 34,000×g for 120 minutes), the pellet was resuspended in MSB.

Protein was estimated by using a protein assay for microsomes (Laboratories, Inc., Hercules, Calif.) and a BCA protein assay kit (Pierce, Thermo Fisher Scientific, Inc., Waltham, Mass.) for lysates with bovine serum albumin as the standard. 60 μg and 40 μg of total lysate and microsomes, respectively, were separated on 10% sodium dodecyl sulfate-polyacrylamide gel electrophoresis (SDS-PAGE) and immunoblotted with 1:10000 dilution of monoclonal anti-CYP3A4 K03 (Schuetz et al., 1996. Proc Natl Acad Sci USA 93:4001-4005), or 1:250 dilution of WB-3A5 (Corning, Inc., Corning, N.Y.), followed by 1:10000 dilution of secondary antibodies HRP-conjugated anti-mouse and anti-rabbit (Jackson ImmunoResearch Inc., West Grove, Pa.), respectively. β-Actin protein expression was determined by monoclonal anti-actin (Sigma, St. louis, MO), followed by 1:10000 dilution of secondary antibodies HRP-conjugated anti-mouse. The blot was developed with the ECL Western Blotting Reagents (GE Healthcare Little Chalfont, United Kingdom). Bands on film were optically scanned.

### Midazolam and Tacrolimus Metabolism Assays

HuH-7 and engineered cells were grown to confluence in 12-well BIOCOAT collagen I multi-well plates (Corning, Inc., Corning, N.Y.) for 3-4 weeks in media. Media was refreshed 2-3 times a week. Cells were then overlaid with MATRIGEL matrix (Corning, Inc., Corning, N.Y.) and then induced for three days by adding 100 μM phenytoin sodium (USP), diluted in methanol, and 10 μM rifampicin (Sigma-Aldrich, St. Louis, Mo.), diluted in methanol, in cell culture. Media was changed daily with inducers rifampicin and phenytoin added. On the fourth day, 500 μl of media was added to the cells with 100 μM phenytoin and 10 μM rifampicin with either equal volumes of methanol, as a negative control, or midazolam (MDZ; Cerrilliant Corp., Round Rock, Tex.) diluted in methanol so that MDZ final concentration was 5 μM (1628 ng/mL) in cell culture media. Cells were incubated overnight, and media was collected and assayed for MDZ, 1-OH MDZ and 4-OH MDZ by high performance liquid chromatography mass spectrometry. To determine the metabolic function of the engineered cell lines on tacrolimus (Toronto Research Chemicals Inc., Toronto, Ontario, Canada) the same process except we used six-well collagen coated plates and 1.5 mL with 13 ng/mL tacrolimus reaction volume.

### Detection Methods for Tac, MDZ, 1-OH MDZ, and 4-OH MDZ

Detection and quantification of midazolam, 1-OH midazolam, and 4-OH midazolam in cell culture media was performed using a high-performance liquid chromatograph (1200 Series, Agilent, Santa Clara, Calif.) coupled with a TSQ Quantum triple stage quadrupole mass spectrometer (Thermo-Electron Corp., San Jose, Calif.). Detection and quantification of tacrolimus was performed using chromatographic separation (Agilent 1100—High Performance Liquid Chromatography, Agilent, Santa Clara, Calif.) and mass spectrometry (API 4000, Sciex Inc., Redwood City, Calif.).

### CYP3CIDE Experiments

CYP3CIDE (Sigma-Aldrich, St. Louis, Mo.) was used as a selective CYP3A4 inhibitor in cell culture and diluted in DMSO. To determine the concentration of CYP3CIDE to use in cell culture, a dose response assay was performed in Huh-7 and the CYP3A5 *1/*1 dd cell line between 100 nM and 1 mM in cell culture using MDZ as the substrate. For further analysis, 50 μM CYP3CIDE was used in experiments with all cell lines. Dose response curves were assessed using PRISM software (Graphpad Software, La Jolla, Calif.).

### Statistical Analysis

All comparisons were conducted using t-test for continuous variables.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and nut as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for making a cell line that metabolizes a drug in a manner similar to a human subject in which genotype of the human subject affects metabolism of the drug, the method comprising:
identifying a locus and allelic variants at the locus in the human subject that affect metabolism of the drug;
providing a parental cell line that comprises the locus that affects metabolism of the drug in the human subject; and
introducing a genetic modification into the parental cell at the locus, thereby forming a variant cell line comprising an allelic variant at the locus having the genetic modification.

2. The method of claim 1, further comprising analyzing metabolism of the drug by the variant cell line compared to a human homozygous for the wild-type allele at the locus.

3. The method of claim 1, further comprising:
analyzing metabolism of the drug by the variant cell line compared to a human subject possessing at least one non-wild-type allele at the locus.

4. The method of claim 3, further comprising:
identifying the cell line as a model cell line approximating metabolism of the drug by a human possessing at least one non-wild-type allele at the locus.

5. The method of claim 1, further comprising:
introducing a second genetic modification into the parental cell line at the locus, thereby producing a second variant cell line that differs from the first variant cell line.

6. The method of claim 5, further comprising analyzing metabolism of the drug by the second variant cell line.

7. The method of claim 6, further comprising identifying the second variant cell line as a model cell line approximating metabolism of the drug by a human possessing at least one non-wild-type allele at the locus.

8. The method of claim 5, further comprising:
introducing a third genetic modification into the parental cell line at the locus, thereby producing a third variant cell line that differs from the first variant cell line and the second variant cell line.

9. (canceled)

10. (canceled)

11. The method of claim 4, further comprising:
increasing the dose of the drug to a patient having the at least one non-wild-type allele at the locus if the cell line metabolizes the drug more rapidly than a human homozygous for the wild-type allele at the locus.

12. The method of claim 4, further comprising:
decreasing the dose of the drug to a patient having the at least one non-wild-type allele at the locus if the cell line metabolizes the drug more slowly than a human homozygous for the wild-type allele at the locus.

13. The method of claim 1, wherein the parental cell comprises an immortal cell line.

14. The method of claim 1, wherein the parental cell is, or is derived from, a human cell.

15. The method of claim 14, wherein the human cell is a liver cell, an intestinal cell, a kidney cell, stomach cell, colorectal cell, pancreatic cell, bile duct cell, urinary tract cell, or nervous system cell.

16. The method of claim 1, wherein the locus is a locus that influences Cytochrome P450 (CYP) activity.

17-19. (canceled)

20. The method of claim 1 further comprising introducing a second genetic modification into the variant cell line at a second locus, thereby producing a second variant cell line that differs from the first variant cell line.

21. The method of claim 20, further comprising analyzing metabolism of the drug by the second variant cell line compared to a human homozygous for the wild-type allele at the locus.

22-24. (canceled)

25. The method of claim 23, further comprising:
decreasing the dose of the drug to a patient having the at least one non-wild-type allele at the locus if the second variant cell line metabolizes the drug more slowly than a human homozygous for the wild-type allele at the locus.

26. A method of providing personalized dosing of a drug to a patient whose genotype at a locus affects metabolism of the drug, the method comprising:
determining the genotype of the patient at the locus;
providing a model cell line comprising a genetic modification at the locus, the genetic modification causing the model cell line to approximate metabolism of the drug by the patient;
culturing the cell line in the presence of the drug;
evaluating metabolism of the drug by the cell line; and
administering to the patient a dose of the drug as determined by metabolism of the drug by the cell line.

27. (canceled)

28. (canceled)

29. A method of determining personalized dosing of a drug to a patient whose genotypes at a first locus and a second locus affect metabolism of the drug, the method comprising:
determining the genotype of the patient at the first locus;
determining the genotype of the patient at the second locus;
providing a cell line comprising:
a first genetic modification at the first locus, and
a second genetic modification at the second locus, so that the cell line approximates metabolism of the drug by the patient;
culturing the cell line in the presence of the drug;
evaluating metabolism of the drug by the cell line; and
determining from the metabolism of the drug by the cell line the dose of the drug effective to treat the patient.

30. A cell line deposited at the American Type Culture Collection (ATCC) with the ATCC Accession No. PTA-123710.

31. A cell line deposited at the American Type Culture Collection (ATCC) with the ATCC Accession No. 124693.

32. A cell line deposited at the American Type Culture Collection (ATCC) with the ATCC Accession No. 124694.
